SEQUENCE LISTING (16 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 40 aa, Homo sapiens
EGTFISDYSIAMDKIHQQDFVNWLLAQKGKKNDWKHNITQ

SEQ ID NO: 3 - protein, 28 aa, Homo sapiens
EGTFISDYSIAMDKIHQQDFVNWLLAQK

SEQ ID NO: 4 - protein, 39 aa, synthetic construct
GTFISDYSIAMDKIHQQDFVNWLLAQKGKKNDWKHNITQ

SEQ ID NO: 5 - protein, 38 aa, synthetic construct
TFISDYSIAMDKIHQQDFVNWLLAQKGKKNDWKHNITQ

SEQ ID NO: 6 - protein, 27 aa, synthetic construct
GTFISDYSIAMDKIHQQDFVNWLLAQK

SEQ ID NO: 7 - protein, 26 aa, synthetic construct
TFISDYSIAMDKIHQQDFVNWLLAQK

SEQ ID NO: 8 - protein, 25 aa, synthetic construct
FISDYSIAMDKIHQQDFVNWLLAQK

SEQ ID NO: 9 - protein, 24 aa, synthetic construct
ISDYSIAMDKIHQQDFVNWLLAQK

SEQ ID NO: 10 - protein, 12 aa, synthetic construct
GKKNDWKHNITQ

SEQ ID NO: 11 - protein, 39 aa, Heloderma suspectum
HGEGTFTSDLSKQMEEEAVRLFIEWLKNGGPSSGAPPPS

SEQ ID NO: 12 - protein, 223 aa, synthetic construct
QVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 214 aa, synthetic construct
EIVMTQSPATLSVSPGERATLSCRASQSVSSNLAWYQQKPGQAPRLLIYGAATRATGIPARVSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 227 aa, synthetic construct
QVQLQQSGAEVKKPGSSVKVSCKASGGTFSSYAISWVRQAPGQGLEWMGGIIPTFGTANYAQKFQGRVTITADESTSTAYMELSSLRSEDTAVYYCAQGPIVGAPTDYWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 15 - protein, 215 aa, synthetic construct
SYVLTQPPSASGTPGQRVAISCSGSNSNIGSNTVHWYQQLPGAAPKLLIYSNNQRPSGVPDRFSGSNSGTSASLAISRLQSEDEADYYCAAWDDSLNGVVFGGGTKVTVLQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 27 aa, synthetic construct
TFISDYSIAMDKIHQQDFVNWLLAQKG

(19) United States
(12) Patent Application Publication
(10) Pub. No.: US 2026/0285992 A1
(43) Pub. Date: Sep. 24, 2026

(54) GIP ACTIVITY MODULATORS AND ORTHOSTATIC INTOLERANCE

(71) Applicant: Antag Therapeutics ApS, Copenhagen N (DK)

(72) Inventor: Alexander Hovard SPARRE-ULRICH, Copenhagen (DK)

(21) Appl. No.: 19/168,687

(22) PCT Filed: Apr. 5, 2024
(86) PCT No.: PCT/EP2024/059332

§ 371 Date: Sep. 24, 2025

(30) Foreign Application Priority Data
Apr. 5, 2023 (EP) 23166737.9

(51) Int. Cl. C07K 16/28; A61K 38/22; A61K 38/26; A61K 38/31; C07K 16/26; C12N 15/113

(52) U.S. Cl. CPC C07K 16/2869; A61K 38/22; A61K 38/26; A61K 38/31; C07K 16/26; C12N 15/1136; C12N 15/1138; C12N 2310/11; C12N 2310/14

(57) ABSTRACT

The present disclosure provided glucose-dependent insulinotropic polypeptide (GIP) activity modulators for use in the treatment of orthostatic intolerance.

### TECHNICAL FIELD

The present invention relates to glucose-dependent insulinotropic polypeptide (GIP) activity modulators, such as glucose-dependent insulinotropic polypeptide receptor (GIPR) antagonists and agonists, for use in treatment of orthostatic intolerance such as orthostatic intolerance with orthostatic hypotension and/or orthostatic tachycardia.

### BACKGROUND

Glucose-dependent insulinotropic polypeptide (GIP) is a hormone of the incretin family and is secreted from the gut following a meal. GIP is also known as gastric inhibitory peptide or polypeptide or gastroinhibitory peptide. Like its sister hormone glucagon-like peptide 1 (GLP-1), GIP is a potent insulin secretagogue. In addition to its insulinotropic actions, GIP has several important roles in human physiology including its involvement in glucagon secretion, fat metabolism, and blood flow in adipose tissue. For these reasons, there are currently treatment strategies involving targeting the GIP system in clinical development for obesity. Interestingly, both agonism and antagonism of the GIP system are being employed in these treatment strategies as pharmacological agonism of the GIPR may result in functional antagonism due to internalization of the GIPR.

Postural haemodynamic homeostasis in humans involves complex adaptive mechanisms controlled by the autonomic nervous system. On standing, as the gravitational challenge abruptly increases, cardiovascular reflexes are initiated to maintain stable blood pressure (BP) and perfusion in the upper body. Conversely, impairment of the compensatory response to standing upright (orthostasis) can lead to a temporary or sustained fall in BP, termed orthostatic hypotension (OH). If the cerebral circulation becomes critically compromised by OH, susceptible individuals may experience chronic fatigue, blurred vision, dizziness, pain in the neck and shoulders (‘coat hanger’ pain) and fainting (syncope) or feeling about to faint (presyncope) (Fedorowski and Melander 2012). Orthostatic instability may induce syncopal attacks either alone or in combination with other mechanisms, and is often dismissed as a precipitating factor.

Orthostatic intolerance (OI) refers to the inability to remain upright without symptoms. This inability can be associated with cardiovascular changes such as a decrease in blood pressure in orthostatic hypotension or tachycardia. The clinical variants of orthostatic hypotension include initial orthostatic hypotension, classical orthostatic hypotension and delayed orthostatic hypotension.

Postural Orthostatic Tachycardia Syndrome (POTS) is a debilitating syndrome that primarily affects women in the childbearing age and it is estimated that around 3 million people suffer from the syndrome in the United States alone. Patients affected by POTS endure a low quality of life due to chronic orthostatic intolerance with symptoms such as dizziness, light-headedness, brain fog, headache, and tachycardia while standing. In the majority of patients with POTS, these symptoms are exacerbated after a meal. As a result of the significant impairment of their quality of life due to the abovementioned symptoms, about 25% of patients file for disability. One of the main drivers of the orthostatic tachycardia in patients with POTS is excessive sympathetic activation, which mostly occurs in response to pooling of blood in the splanchnic veins upon standing.

There is a highly unmet medical need for the development of improved treatment options for patients affected by orthostatic intolerance as there are not pharmacological options.

### SUMMARY

The present disclosure relates to a GIP (glucose-dependent insulinotropic polypeptide) activity modulator for use in the treatment of orthostatic intolerance.

In some embodiments said GIP activity modulator is a GIPR activity modulator.

In some embodiments said GIP activity modulator is a modulator of GIP secretion

In some embodiments said GIP activity modulator is a GIP or GIPR inhibitor.

In some embodiments said GIP activity modulator is a GIP or GIPR antibody.

In some embodiments said GIP activity modulator is a GIPR antagonist.

In some embodiments said GIP activity modulator is a GIPR agonist.

### DESCRIPTION OF DRAWINGS

FIG. 1. The effect of GIP or placebo administration on supine and orthostatic heart rate. The heart rates of four healthy subjects (1 male and 3 females) were measured in the supine position and during orthostatic challenges (OC) before and after subcutaneously administered GIP (1-42) or placebo at time 0 min. Data presented as meantsem change from pre-injection supine heart rate.

FIG. 2. The effect of GIP or placebo administration on supine and orthostatic heart rate measured by a Holter monitor. The heart rates of a healthy subject were measured in the supine position and during orthostatic challenges (OC) using a Holter monitor before and after subcutaneously administered GIP (1-42) or placebo at time 0 min. Data presented as change from pre-injection supine heart rate.

FIG. 3. The effect of GIP or placebo administration on supine and orthostatic heart rate measured by a Holter monitor. The heart rates of a healthy subject were measured in the supine position and during orthostatic challenges (OC) using a Holter monitor before and after subcutaneously administered GIP (1-42) or placebo at time 0 min. Data presented as change from pre-injection supine heart rate.

FIG. 4. The effect of GIP or placebo administration on supine and orthostatic systolic blood pressure (SBP). The SBP of four healthy subjects (1 male and 3 females) were measured in the supine position and during orthostatic challenges (OC) before and after subcutaneously administered GIP (1-42) or placebo at time 0 min. Data presented as meantsem change from pre-injection supine SBP.

FIG. 5. The effect of GIP or placebo administration on supine and orthostatic diastolic blood pressure (DBP). The DBP of four healthy subjects (1 male and 3 females) were measured in the supine position and during orthostatic challenges (OC) before and after subcutaneously administered GIP (1-42) or placebo at time 0 min. Data presented as meantsem change from pre-injection supine DBP.

### DEFINITIONS

The term “agonist” in the present context refers to a compound capable of binding to and activating downstream signaling cascades from a receptor.

The term “antagonist” as used herein refers to a compound capable of binding to and blocking or reducing agonist-mediated responses of a receptor. Antagonists usually do not provoke a biological response themselves upon binding to a receptor. Antagonists have affinity but no efficacy for their cognate receptors, and binding of an antagonist to its receptor will inhibit the function of an agonist or inverse agonist at the receptor.

Antagonists mediate their effects by binding to the active (orthosteric) site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of drug antagonists typically achieve their potency by competing with endogenous ligands or substrates at structurally defined binding sites on receptors. Antagonists may be competitive, non-competitive, uncompetitive, silent antagonists, partial agonists or inverse agonists.

A competitive antagonist (also known as surmountable antagonist) reversibly binds to receptors at the same binding site (i.e. at the active site) as the endogenous ligand or agonist, but without activating the receptor. Agonists and antagonists thus “compete” for the same binding site on the receptor. Once bound, an antagonist blocks agonist binding. The level of receptor activity is determined by the relative affinity of each ligand for the binding site and the relative concentrations of the ligands. High concentrations of a competitive antagonist will increase the proportion of receptors occupied by the antagonist.

The term “non-competitive antagonism” (also called nonsurmountable or insurmountable antagonism) describes two distinct phenomena with functionally similar results: one in which the antagonist binds to the active site of the receptor, and one in which the antagonist binds to an allosteric site of the receptor. Unlike competitive antagonists, which affect the amount of agonist necessary to achieve a maximal response but do not affect the magnitude of that maximal response, non-competitive antagonists reduce the magnitude of the maximum response that can be attained by any amount of agonist.

The term “silent antagonist” refers to a competitive receptor antagonist that has absolutely no intrinsic activity for activating a receptor.

The term “partial agonist” refers to an agonist that, at a given receptor, might differ in the amplitude of the functional response that it elicits after maximal receptor occupancy. Partial agonists can act as a competitive antagonist in the presence of a full agonist (or a more efficacious agonist), as it competes with the full agonist for receptor occupancy, thereby producing a net decrease in the receptor activation as compared to that observed with the full agonist alone.

The term “inverse agonist” refers to a ligand that is capable of binding to the same receptor binding site as an agonist and antagonize its effects. Furthermore, an inverse agonist can also inhibit the basal activity of constitutively active receptors.

The term “GIP activity modulator” as used herein may refer to a compound which
binds the GIP peptide and blocks its interaction with the GIPR;
binds the GIP peptide and stimulates its interaction with the GIPR;
is an antagonist of the GIPR;
is an agonist of the GIPR;
increases GIP secretion;
reduces GIP secretion;
or otherwise interacts with, activates or inhibits GIP or GIPR.

The term “GIPR activity modulator” as used herein may refer to a compound which is an antagonist of a GIPR or an agonist of a GIPR.

Thus, the term “GIP activity modulator” as used herein also encompasses “GIPR activity modulators”.

Glucose-dependent insulinotropic polypeptide or Glucose-dependent insulinotropic peptide is used interchangeably herein, and both are abbreviated GIP.

The term “glucose-dependent insulinotropic polypeptide receptor (GIPR) antagonist” as used herein refers to a compound, such as a peptide, capable of binding to and blocking and/or reducing agonist-mediated responses of GIPR; and/or blocking and/or reducing basal activity of GIPR (non-agonist mediated/constitutive active GIPR).

The term “glucose-dependent insulinotropic polypeptide receptor (GIPR) agonist” as used herein refers to a compound, such as a peptide, capable of binding to and activating downstream signaling cascades from a GIPR.

An “isolated peptide” is a peptide separated and/or recovered from a component of their natural, typically cellular, environment, that is essentially free from contaminating cellular components, such as carbohydrate, lipid, or other proteinaceous impurities associated with the polypeptide in nature. Typically, a preparation of isolated peptide contains the peptide in a highly purified form, i.e., at least about 80% pure, at least about 90% pure, at least about 95% pure, greater than 95% pure, or greater than 99% pure. The term “isolated” does not exclude the presence of the same peptide in alternative physical forms, such as dimers, tetramers or alternatively glycosylated or derived forms.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3557-59 (1968) 18 and adopted in 37 C.F.R., section 1.822 (b) (2) belong to the group of amino acids listed herewith: Y, G, F, M, A, S, I, L, T, V, P, K, H, Q, E, W, R, D, N and C. Non-natural amino acids are those not listed immediately above. Also, non-natural amino acid residues include, but are not limited to, modified amino acid residues, L-amino acid residues, and stereoisomers of D-amino acid residues.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

Within the meaning of the term “equivalent amino acid substitution” as applied herein, one amino acid may be substituted for another, in one embodiment, within the groups of amino acids indicated herein below:
i) Amino acids having polar side chains (Asp, Glu, Lys, Arg, His, Asn, Gln, Ser, Thr, Tyr, and Cys)
ii) Amino acids having non-polar side chains (Gly, Ala, Val, Leu, Ile, Phe, Trp, Pro, and Met)
iii) Amino acids having aliphatic side chains (Gly, Ala Val, Leu, Ile)
iv) Amino acids having cyclic side chains (Phe, Tyr, Trp, His, Pro)
v) Amino acids having aromatic side chains (Phe, Tyr, Trp)
vi) Amino acids having acidic side chains (Asp, Glu)
vii) Amino acids having basic side chains (Lys, Arg, His)
viii) Amino acids having amide side chains (Asn, Gln)
ix) Amino acids having hydroxy side chains (Ser, Thr)
x) Amino acids having sulphur-containing side chains (Cys, Met),
xi) Neutral, weakly hydrophobic amino acids (Pro, Ala, Gly, Ser, Thr)
xii) Hydrophilic, acidic amino acids (Gln, Asn, Glu, Asp), and
xiii) Hydrophobic amino acids (Leu, Ile, Val)

Where the L or D form (optical isomers) has not been specified it is to be understood that the amino acid in question has the natural L form, cf. Pure & Appl. Chem. Vol. (56 (5) pp 595-624 (1984) 19 or the D form, so that the peptides formed may be constituted of amino acids of L form, D form, or a sequence of mixed L and D forms.

A “functional variant” of a peptide is a peptide capable of performing essentially the same functions as the peptide it is a functional variant of. In particular, a functional variant can essentially bind the same molecules, such as receptors, or perform the same receptor mediated responses as the peptide it is a functional variant of. A functional variant may comprise one or more equivalent amino acid residues.

The term “functional fragment” when used in reference to a polypeptide means a polypeptide that is truncated, i.e., missing one or more amino acids, and that retains one or more desired activities. In particular, a functional fragment may retain affinity to receptors of the peptide that it is a functional variant of, but without activating the receptor.

A “bioactive agent” (i.e. a biologically active substance/agent) is any agent, drug, compound, composition of matter or mixture which provides some pharmacologic, often beneficial, effect that can be demonstrated in vivo or in vitro. It refers to the GIP activity modulators and GIP peptide analogues as defined herein and compounds or compositions comprising these. As used herein, this term further includes any physiologically or pharmacologically active substance that produces a localized or systemic effect in an individual.

The terms “drug” and “medicament” as used herein include biologically, physiologically, or pharmacologically active substances that act locally or systemically in the human or animal body.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the compound or composition according to the present disclosure for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering compositions of the present invention. In one embodiment, a treatment according to the present invention can be prophylactic, ameliorating and/or curative. The individual to be treated is preferably a mammal, in particular a human being. Treatment of animals, such as mice, rats, dogs, cats, cows, horses, sheep, apes and pigs, is, however, also encompassed herewith. As used herein, ‘subject’ and ‘individual’ may be used interchangeably.

An “individual in need thereof” refers to an individual who may benefit from the treatments of the present disclosure. In one embodiment, said individual in need thereof is a diseased individual, wherein said disease is orthostatic intolerance, such as orthostatic intolerance with orthostatic hypotension and/or orthostatic tachycardia.

“Orthostatic intolerance” (OI) refers to the development of symptoms in the upright posture, particularly standing upright, that are relieved when reclining. Orthostatic intolerance encompass orthostatic hypotension, orthostatic reduction in blood pressure, orthostatic tachycardia, orthostatic increase in heart rate, syncope and presyncope, dizziness and light-headedness, in fasting (postprandial) or fed conditions.

Tachycardia is the medical term for a heart rate over 100 beats per minute (bpm).

“Orthostatic hypotension” (OH) refers to a decrease in blood pressure that occurs soon after moving from a laying down (supine) position to standing up (upright) position or to a sitting up position, or tilting up the head of at least 60°. Clinically, orthostatic hypotension is associated with the presence of at least one of the following: a decrease in systolic blood pressure of ≥20 mm Hg; a decrease in diastolic blood pressure of ≥10 mm Hg; and/or a decrease in systolic blood pressure to an absolute value of <90 mmHg; each within at least 40 seconds from the change in position (initial OH), within 3 minutes from the change in position (classical OH), or after 3 minutes from the change in position (delayed OH).

“Pharmacologically effective amount”, “pharmaceutically effective amount” or “physiologically effective amount” of a bioactive agent is the amount of a bioactive agent present in a pharmaceutical composition as described herein that is needed to provide a desired level of active agent in the bloodstream or at the site of action in an individual (e.g. the lungs, the gastric system, the colorectal system, prostate, etc.) to be treated to give an anticipated physiological response when such composition is administered. A bioactive agent in the present context refers to a GIP activity modulator as disclosed herein.

“Co-administering” or “co-administration” as used herein refers to the administration of one or more GIP activity modulators of the present disclosure and a state-of-the-art pharmaceutical composition. The at least two components can be administered separately, sequentially or simultaneously.

### DETAILED DESCRIPTION

GIP refers to glucose-dependent insulinotropic polypeptide. As used herein the abbreviation GIP or hGIP is human GIP (Uniprot accession number P09681). GIP is derived from a 153-amino acid proprotein and circulates as a biologically active 42-amino acid peptide.

The sequence of native hGIP1-42 is (SEQ ID NO: 1):

YAEGTFISDYSIAMDKIHQQDFVNWLLAQKGKKNDWKHNITQ

GIPR (or GIP receptor) refers to the glucose-dependent insulinotropic polypeptide receptor. This seven-transmembrane protein is found on multiple tissues and cell types. As used herein the abbreviation hGIPR is human GIPR (Uniprot accession number P48546).

### GIP Activity Modulators

The present disclosure relates to a GIP activity modulator for use in the treatment of orthostatic intolerance and/or orthostatic hypotension.

In some embodiments, said GIP activity modulator is a GIPR activity modulator.

In some embodiments, said GIP activity modulator is a modulator of GIP secretion. For example, in some embodiments, said GIP activity modulator increases GIP secretion.

In some embodiments, said GIP activity modulator decreases GIP secretion.

In some embodiments, said GIP activity modulator is a GIPR ligand.

In one embodiments, the GIPR activity modulator is capable of binding to, or binds to, one or more of the hGIPR (Uniprot accession number P48546), the rGIPR (Uniprot accession number P43219), the mGIPR (Uniprot accession number Q0P543), the dog GIPR (Uniprot accession number E2RIK5), the pig GIPR (Uniprot accession number I3LND8), and the Macaca mulatta GIPR (Uniprot accession number A0A1D5QDM0) (primate).

In some embodiments, said GIP activity modulator is a GIP inhibitor.

In some embodiments, said GIP activity modulator is a GIPR inhibitor.

In some embodiments, said GIP activity modulator is a small molecule.

In some embodiments, said GIP activity modulator is a peptide.

In some embodiments, said GIP activity modulator is an agonist of a GIPR.

In some embodiments, said GIP activity modulator is an antagonist of a GIPR.

In one embodiment, the GIP activity modulator is capable of binding to a GIPR.

In one embodiment, the GIP activity modulator is capable of binding to and antagonising a GIPR.

In some embodiments, said GIP activity modulator is an antagonist of the hGIPR or an agonist of hGIPR.

In one embodiment the GIP activity modulator disclosed herein is a competitive antagonist of the hGIP receptor.

In some embodiments, the GIP activity modulator is an antagonist or an agonist of a GIPR selected from the group consisting of the human GIPR (Uniprot accession number P48546), the mouse GIPR (Uniprot accession number QOP543), the rat GIPR (Uniprot accession number P43219), the dog GIPR (Uniprot accession number E2RIK5), the pig GIPR (Uniprot accession number I3LND8), and/or the Macaca mulatta GIPR (Uniprot accession number A0A1D5QDM0) (primate).

In some embodiments, said GIP activity modulator is an antibody. In some embodiments, said GIP activity modulator is an anti-GIP antibody or an anti-GIPR antibody.

In some embodiments, said GIP activity modulator is a GIP peptide analogue. In one embodiment, the GIP activity modulator is a GIP peptide analogue capable of binding to a GIPR. In one embodiment, the GIP activity modulator is a GIP peptide analogue capable of binding to and antagonising a GIPR (a GIPR antagonist). In one embodiment, the GIP activity modulator comprises a GIP peptide analogue capable of binding to and promoting activity of a GIPR (a GIPR agonist).

A peptide as defined herein includes native peptide sequences and also functional variants and functional fragments of the defined amino acid sequences of said peptide. ‘Identity’ and ‘sequence identity’ may be used interchangeably herein. The terms ‘variant’ and ‘functional variant’ may be used interchangeably herein.

### GIP/GIPR Antibodies

In some embodiments, the GIP activity modulator of the present disclosure is an antibody.

In some embodiments, the GIP activity modulator of the present disclosure is an anti-GIPR antibody and/or an anti-GIPR antibody fragment. In some embodiments said anti-GIPR antibody is an antagonist of the GIPR.

In some embodiments, the GIP activity modulator of the present disclosure is a GIPR antibody and/or a GIPR antibody fragment. In some embodiments said GIPR antibody is an agonist of the GIPR.

In one embodiments, the GIP activity modulator is an anti-GIPR antibody and/or anti-GIPR antibody fragment capable of binding to, or which binds to, one or more of the hGIPR (Uniprot accession number P48546), the rGIPR (Uniprot accession number P43219), the mGIPR (Uniprot accession number Q0P543), the dog GIPR (Uniprot accession number E2RIK5), the pig GIPR (Uniprot accession number I3LND8), and the Macaca mulatta GIPR (Uniprot accession number A0A1D5QDM0) (primate).

In some embodiments, said GIP activity modulator is an anti-GIP antibody and/or an anti-GIP antibody fragment. In some embodiments said anti-GIP antibody binds to GIP. In some embodiments said anti-GIP antibody is capable of interfering with, such as inhibiting, the binding of GIP to the GIPR.

In some embodiments, said GIP activity modulator is an anti-GIP antibody and/or an anti-GIP antibody fragment, wherein said antibody and/or antibody fragment may be conjugated to a moiety that binds to a target other than GIP and GIPR.

In some embodiments, said GIP activity modulator is a polyclonal anti-GIP antibody. In some embodiments, said GIP activity modulator is a monoclonal anti-GIP antibody.

In some embodiments, said GIP activity modulator is an antibody or functional fragment thereof that specifically binds to human GIPR, wherein the antibody or functional fragment thereof comprises a cysteine or non-canonical amino acid amino acid substitution at one or more conjugation site(s) as disclosed and defined in WO18136440.

In some embodiments, said GIP activity modulator is a monoclonal antibody or an antibody fragment as disclosed in WO17112824.

In some embodiments, said GIP activity modulator is a monoclonal antibody or an antibody fragment as disclosed in WO21092545.

In some embodiments, said GIP activity modulator is an antibody or an antibody fragment as described in WO18064307 A2.

In some embodiments, said GIP activity modulator is an antibody or an antibody fragment GIPR antagonist as described WO20185533 A1.

In some embodiments, said GIP activity modulator is a monoclonal antibody or an antibody fragment that specifically binds to hGIPR as disclosed in WO18237097 A1.

In some embodiments, the GIP activity modulator is an antibody or a functional fragment of the antibody as disclosed in Killion E A, Wang J, Yie J, et al “Anti-obesity effects of GIPR antagonists alone and in combination with GLP-1R agonists in preclinical models” Sci Transl Med, 2018; 10 (472).

In some embodiments, the GIP activity modulator is an antibody or antibody fragment which comprises a functional fragment of the heavy chain as set forth in SEQ ID NO: 12;

(QVQLVESGGGVVQPGRSLRLSCAASGFTFSNYGMHWVRQAPGEG
LEWVAAIWFDASDKYYADAVKGRFTISRDNSKNTLYLQMNSLRAE
DTAVYYCARDQAIFGVVPDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSC)

and the light chain as set forth in SEQ ID NO: 13:

(EIVMTQSPATLSVSPGERATLSCRASQSVSSNLAWYQQKPGQAP
RLLIYGAATRATGIPARVSGSGSGTEFTLTISSLQSEDFAVYYCQ
QYNNWPLTFGGGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC).

In some embodiments, said GIP activity modulator is a polyclonal anti-GIPR antibody.

In some embodiments, said GIP activity modulator is a monoclonal anti-GIPR antibody.

In some embodiments, the GIP activity modulator is an antibody or a functional fragment of the antibody GIPG013 as disclosed in Ravn P, Madhurantakam C, Kunze S, et al “Structural and pharmacological characterization of novel potent and selective monoclonal antibody antagonists of glucose-dependent insulinotropic polypeptide receptor” J Biol Chem, 2013; 288 (27): 19760-19772.

In some embodiments, the GIP activity modulator is an antibody or antibody fragment which is a functional fragment of the heavy chain as set forth in SEQ ID NO: 14:

(QVQLQQSGAEVKKPGSSVKVSCKASGGTFSSYAISWVRQAPGQG
LEWMGGIIPTFGTANYAQKFQGRVTITADESTSTAYMELSSLRSE
DTAVYYCAQGPIVGAPTDYWGKGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKRVEPKSCDK
THT)

and the light chain as set forth in SEQ ID NO: 15:

(SYVLTQPPSASGTPGQRVAISCSGSNSNIGSNTVHWYQQLPGAA
PKLLIYSNNQRPSGVPDRFSGSNSGTSASLAISRLQSEDEADYYC
AAWDDSLNGVVFGGGTKVTVLQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECS).

In some embodiments, the GIP activity modulator of the present disclosure is an antigen binding protein, an antibody or an antibody fragment according to the disclosures selected from the group consisting of WO15095354 A2; WO15095354 A3; WO18102654 A1; WO20014329 A1; WO18124010 A1; WO21052349 A1; WO21092545 A2; WO18136440 A1; WO18169954 A1; WO17112824 A2; WO18124011 A1; WO18124009 A1; WO21202013 A1; WO16005643 A1; WO18064307 A2; WO20185533 A1; WO19179424 A1; CN114231493 A; WO21196225 A1; WO18237097 A1; WO16104439 A1; WO18237095 A1; JP2013138638 A2; WO11014680 A2; WO11014680 A3; WO06119905 A1; WO9840477 A1; WO06086823 A1; WO9824464 A1; WO03103697 A2; and WO0020592 A1.

Mono and Dual GIP activity modulators

In some embodiments, the GIP activity modulator is a modulator of GIP and/or GIPR. In some embodiments, the GIP activity modulator is an agonist or an antagonist of the GIPR. In some embodiments, the GIP activity modulator is an agonist or an antagonist of the GIPR and one or more targets such as protein targets.

In some embodiments, the GIP activity modulator of the present disclosure targets at least GIP (via GIP or the GIPR). In some embodiments, the GIP activity modulator of the present disclosure targets both GIP (via GIP or the GIPR) as well as one or more additional targets. In some embodiments, said GIP activity modulator is a combined GIP activity modulator and a modulator of one or more additional targets, such as protein targets. These may be denoted dual modulators or triple modulators, such as dual agonists/antagonists or triple agonists/antagonists.

Incretins are gut-derived hormones, members of the glucagon superfamily, released in response to nutrient ingestion, mainly glucose and fat. They stimulate pancreatic insulin secretion in a glucose-dependent manner and play an important role in the local gastrointestinal and whole-body physiology.

In some embodiments said GIP activity modulator targets GIP as well as one or more incretins, such as one or more incretins selected from GLP-1 and GLP-2. In some embodiments said GIP activity modulator is a GIP activity modulator and an incretin modulator, such as a GLP-1 activity modulator and/or a GLP-2 activity modulator. In some embodiments said GIP activity modulator targets GIPR as well as one or more incretin receptors, such as one or more incretin receptors selected from GLP-1R and GLP-2R. In some embodiments said GIP activity modulator is a GIPR activity modulator and a GLP-1R activity modulator and/or a GLP-2R activity modulator.

In some embodiments said GIP activity modulator targets GIP as well as one or more of a GLP-1/GLP-1R modulator, a GLP-2/GLP-2R modulator, a glucagon/glucagon receptor modulator and a Cholecystokinin (CCK) modulator.

In some embodiments said GIP activity modulator is an agonist of GIPR and an agonist or an antagonist of one or more additional incretin receptors, such as one or more incretin receptors selected from the group consisting of GLP-1R and GLP-2R.

In some embodiments said GIP activity modulator is an antagonist of GIPR and an agonist or an antagonist of one or more additional incretin receptors, such as one or more incretin receptors selected from the group consisting of GLP-1R and GLP-2R.

In some embodiments said GIP activity modulator is an agonist of GIPR and an agonist of GLP-1R. Dual GIP/GLP-1 agonists are known in the art, such as tirzepatide (Fisman & Tenembaum 2021, Cardiovascular Diabetology 20, 225).

In some embodiments said GIP activity modulator is an agonist of GIPR and an agonist of GLP-2R.

In some embodiments said GIP activity modulator is an agonist of GIPR and an agonist of GLP-1R and GLP-2R.

In some embodiments, said GIP activity modulator is a GIP-GLP-1 dual agonist as disclosed in WO13164483 A1.

In some embodiments said GIP activity modulator is a GIPR activity modulator and a glucagon activity modulator. In some embodiments said GIP activity modulator is a GIPR agonist and a glucagon receptor agonist.

In some embodiments said GIP activity modulator is a GIPR agonist and an agonist of one or more of the GLP-1R, GLP-2R and glucagon receptor.

In some embodiments the GIP activity modulator of the present disclosure is an agonist or an antagonist according to the disclosures selected from the group consisting of: WO17074714 A1; WO17074715 A1; WO15095354 A2; WO18102654 A1; WO20014329 A1; WO21203864 A1; WO18124010 A1; WO17116204 A1; WO17116205 A1; WO21193983 A2; WO21021877 A1; WO21260530 A1; WO21066600 A1; WO21094259 A1; WO19173787 A1; WO20096695 A1; WO20097394 A1; WO21034727 A1; WO21034728 A1; WO21068251 A1; WO21193984 A2; WO23277620 A1; WO21150673 A1; WO21239082 A1; WO07073486 A2; WO09158704 A2; WO20263063 A1; WO21145552 A1; WO22080991 A1; WO20263063 A1; WO21145552 A1; WO22080991 A1; WO21221482 A1; WO21235916 A1; WO22272018 A1; WO16108586 A1; WO21010532 A1; WO13164483 A1; WO22139538 A1; WO22257979 A1; WO15003122 A2; WO21215801 A1; WO21211976 A2; WO22066212 A1; WO22029231 A1; WO21198229 A1; WO16081884 A2; WO20023388 A1; WO21129894 A1; WO21037212 A1; WO17112824 A2; WO17116936 A1; WO11131371 A1; WO12095303 A1; WO13056852 A1; WO13104539 A1; WO13104540 A1; WO19101035 A1; WO15038938 A1; WO15095406 A1; WO18124011 A1; WO21107660 A1; WO17109087 A1; WO19211451 A1; WO18124009 A1; WO16111971 A1; WO15186988 A1; WO16066744 A2; WO20023382 A1; WO20159949 A1; WO20092191 A1; WO19245893 A2; WO22199629 A1; WO22121667 A1; WO22121666 A1; WO20130749 A1; WO22178366 A1; WO22177744 A1; WO20207477 A1; WO20207477 A9; WO21093883 A1; WO22079639 A1; WO22159395 A1; WO20244556 A1; WO18100134 A1; WO18104718 A1; WO22111370 A1; WO22035271 A1; WO22035302 A1; WO14192284 A1; WO18225041 A1; WO20185533 A1; WO13130683 A2; WO13130684 A1; CN114437181 A; WO22090447 A1; WO20067557 A2; WO15067715 A2; WO22080987 A1; WO22080986 A1; WO22080989 A1; WO19190291 A1; WO19190293 A1; WO13028989 A1; WO22065899 A1; WO22065897 A1; WO22065898 A1; CN114231493 A; WO16205488 A1; WO20023386 A1; WO20053355 A2; WO20053355 A3; CN114042149 A; WO20067575 A1; WO15067716 A1; WO21196225 A1; WO18237097 A1; WO19193204 A1; WO19229225 A1; CN113150172 A; WO21126990 A1; WO05047297 A1; WO08055940 A2; WO14096145 A1; WO14096148 A1; WO14096149 A1; WO14096150 A1; WO16049190 A1; WO13192130 A1; WO19199642 A1; WO18069442 A1; WO18237095 A1; KR20200131784 A; CN111825758 A; WO14158900 A1; WO19072963 A1; WO15184510 A1; US2017114122 AA; WO17160669 A1; WO10071807 A1; WO10016940 A2; WO11143209 A1; WO11143208 A1; WO12167744 A1; WO19140030 A1; WO10011439 A2; WO12088116 A2; WO09099763 A1; WO15086728 A1; WO13074910 A1; WO12138941 A1; WO06052608 A2; WO06105345 A2; WO06105527 A2; WO07055728 A1; WO07055743 A2; WO07114838 A1; WO13192129 A1; WO12166951 A1; WO18065634 A1; WO14081849 A1; WO15086729 A1; US2016015788 AA; WO15022420 A1; WO15086730 A1; WO10016944 A2; WO16077806 A1; WO10016938 A2; US2017112897 AA; WO10016935; WO11094337 A1; WO16198624 A1; WO16131893 A1; WO12055770 A1; WO14152460 A2; WO16077220 A1; WO15035419 A1; WO09116067 A2; WO07120689 A2; WO14049610 A2; WO13003449 A2; WO0058360 A2; WO05082928 A2; WO12168464 A1; WO14074700 A1; WO04037169 A2; CL2011003173 A1; WO06119905 A1; WO11150032 A1; WO04112701 A2; WO04064778 A2; WO05072045 A2; WO04058266 A1; WO04032836 A2; WO04110436 A1; WO06126695 A1; WO10016936 A1; WO04103276 A2; WO05094886 A1; WO0187341 A1; WO07028633 A2; WO05044195 A2; WO04069162 A2; WO9824464 A1; WO03082817 A2; WO04050022 A2; WO04050022; WO03105760 A2; WO03103697 A2; WO03082898 A2; and DOP2004000842 A.

### Modulating GIP Protein/Expression

In some embodiments, the GIP activity modulator of the present disclosure is capable of modulating the expression of GIP or GIPR.

Two major therapeutic approaches in the field of targeting protein expression are the antisense oligonucleotides (ASOs) that inhibit mRNA translation and the oligonucleotides, which function via RNA interference (RNAi) pathway.

In some embodiments, the GIP activity modulator is an antisense oligonucleotide (ASO) targeting GIP or GIPR.

In some embodiments, the GIP activity modulator is an RNAi oligonucleotide targeting GIP or GIPR.

In some embodiments the GIP activity modulator of the present disclosure is a GIP or GIPR modulator according to the disclosures selected from the group consisting of: WO18124010 A1; WO13164483 A1; WO22248506 A1; WO21198229 A1; WO18124011 A1; WO18104718 A1; CN114231493 A; CN114042149 A; WO22020388 A1; WO12161670 A2; WO21198385 A1; WO17004623 A1; WO15140212 A1; CN107385060 A; KR20190124469 A; CN103965344 A; WO08067759 A1; IN03161MU2015 A; WO08104580 A1; WO05075436 A2; WO05039548 A2; WO04098625 A2; WO04099134 A2; WO04098591 A2; GB2545395 A1; CN106676144 A; CN105219802 A; WO06086769 A2; WO08021560 A2; WO07120689 A2; WO14168153 A1; WO10122367 A2; WO10122506 A2; WO0168828 A2; CN102879767 A; WO06119905 A1; KR101091041 B1; US2009228993 AA; WO03048193 A2; WO07054577 A1; WO05094886 A1; WO05090600 A2; WO9824464 A1; AU2008200852 AA; WO05035720; WO05120474 A2; WO02096195 A1; EP1470256 A2; EP1470256 A4; WO0020592 A1; US2005159379 AA; WO03070968 A2; WO0242413 A2; JP2000342109 A2; EP0979872 A1; WO9625487 A1; EP0479210 B1; EP0269072 B1; EP0269072 A3; and EP0269072 A2.

In some embodiments, the GIP activity modulator of the present disclosure is capable of proteomic modulation of GIP or GIPR. In some embodiments, the GIP activity modulator is capable of post-translational modulation of GIP or GIPR. In some embodiments, the GIP activity modulator is capable of degradation or stabilization of GIP or GIPR. In some embodiments, the GIP activity modulator is capable of inhibiting the activity of GIP or GIPR.

In some embodiments the GIP activity modulator of the present disclosure is a GIP or GIPR modulator according to the disclosures selected from the group consisting of: WO18181864 A1; WO06025882 A2; WO10075465 A1; WO11014797 A1; WO12054500 A2; WO15095389 A1; WO19157099 A1; WO17116204 A1; WO17116205 A1; WO21193983 A2; WO21193984 A2; WO20263063 A1; WO21145552 A1; WO22080991 A1; WO21052349 A1; WO22253202 A1; WO21198229 A1; WO19101035 A1; WO19211451 A1; WO22121667 A1; WO21143810 A1; WO22159395 A1; WO20067557 A2; CN114231493 A; WO20023386 A1; WO20067575 A1; WO16049190 A1; WO18069442 A1; WO14158900 A1; WO19072963 A1; U.S. Pat. No. 10,488,423 BB; JP2019218309 A2; WO10071807 A1; WO11143209 A1; WO11143208 A1; WO12167744 A1; WO10011439 A2; WO09099763 A1; WO13074910 A1; WO12088379 A2; WO12138941 A1; WO16084826 A1; WO10148089 A1; WO11094337 A1; US2013244932 AA; WO14152460 A2; WO11104337 A1; WO06086769 A2; WO08021560 A2; WO0058360 A2; WO05082928 A2; WO08149382 A1; KR101091041 B1; WO04039392 A2; WO0020592 A1; and WO05025554 A2.

### Modulating GIP Secretion/Release

In one embodiment the GIP activity modulator of the present disclosure is capable of modulating GIP secretion and/or release.

In one embodiment the GIP activity modulator of the present disclosure is capable of increasing or stimulating GIP secretion and/or release.

In one embodiment the GIP activity modulator of the present disclosure is capable of reducing gastric emptying.

In one embodiment the GIP activity modulator of the present disclosure is capable of inhibiting GIP secretion and/or release. A compound capable of inhibiting GIP secretion and/or release is GLP-1 and GLP-1 analogues as well as somatostatin. In one embodiment the GIP activity modulator is GLP-1 or a GLP-1 analogue. GLP-1 and GLP-1 analogues are well-known in the art, including semaglutide and liraglutide.

In one embodiment the GIP activity modulator is a GLP-1 receptor agonist. GLP-1R agonists are well-known in the art and include dulaglutide, exenatide, semaglutide and liraglutide.

In some embodiments the GIP activity modulator of the present disclosure is a compound capable of modulating GIP secretion and/or release according to the disclosures selected from the group consisting of: WO18124010 A1; WO13164483 A1; WO18124011 A1; WO15048332 A2; WO15048333 A2; WO15048334 A2; WO15048339 A2; WO15048339 A3; WO15048340 A2; WO15048342 A2; WO15048345 A2; WO15048346 A2; WO15048348 A2; WO18124009 A1; WO16066744 A2; WO16011269 A1; WO17004432 A1; WO20205844 A1; WO21188781 A1; WO15067715 A2; JP2019043858 A2; WO12161670 A2; WO15067716 A1; WO21198385 A1; WO15141493 A1; WO10082689 A2; WO13039210 A1; WO09126719 A2; US2009143329 AA; EP2065046 A1; US2008300647 AA; WO07120689 A2; WO0058360 A2; WO05082928 A2; WO09108621 A2; CN102961388 A; JP2009126837 A2; WO07122801 A1; WO06119905 A1; JP2006342084 A2; JP2006342085 A2; WO0187341 A1; JP2009125028 A2; US2008300646 AA; US2008300645 AA; DE19921537 A1; EP0269072 A2; and EP0270321 A2.

### GIP Peptide Analogues

In some embodiments, the GIP activity modulator is a peptide capable of binding to a GIP receptor.

In some embodiments, the GIP activity modulator for use according to the present disclosure is a GIP peptide analogue of sequence hGIP (1-42) as set forth in SEQ ID NO: 1, or a functional variant thereof having at least 75% sequence identity to said hGIP (1-42). In some embodiments said functional variant has least 80% sequence identity to said hGIP (1-42). In some embodiments said functional variant has least 85% sequence identity to said hGIP (1-42). In some embodiments said functional variant has least 90% sequence identity to said hGIP (1-42). In some embodiments said functional variant has least 95% sequence identity to said hGIP (1-42).

In some embodiments, the GIP activity modulator for use according to the present disclosure is a GIP peptide analogue of sequence hGIP (1-42) as set forth in SEQ ID NO: 1, or a functional variant thereof having 1 to 6 individual amino acid substitutions compared to said hGIP (1-42).

In some embodiments, the GIP activity modulator for use according to the present disclosure is a GIP peptide analogue which is a functional fragment of hGIP (1-42) (SEQ ID NO:1) wherein said functional fragment comprises 20 to 40 amino acids of said GIP (1-42); and is capable of binding a GIPR.

In some embodiments, the GIP activity modulator for use according to the present disclosure is a GIP peptide analogue of sequence hGIP (3-30) as set forth in SEQ ID NO: 3, or a functional variant thereof having at least 70% sequence identity to said hGIP (3-30) as set forth in SEQ ID NO: 3.

In some embodiments, the GIP activity modulator for use according to the present disclosure is a non-native GIP peptide.

In some embodiments, the GIP activity modulator for use according to the present disclosure is a GIP peptide analogue having an amino acid sequence that differs from a native GIP peptide, such as differs in having one or more amino acid substitutions.

In some embodiments, said functional variant has at least 70% sequence identity to said hGIP (3-30) as set forth in SEQ ID NO: 3.

In some embodiments, said functional variant has at least 75% sequence identity to said hGIP (3-30) as set forth in SEQ ID NO: 3.

In some embodiments, said functional variant has at least 80% sequence identity to said hGIP (3-30) as set forth in SEQ ID NO: 3.

In some embodiments, said functional variant has at least 85% sequence identity to said hGIP (3-30) as set forth in SEQ ID NO: 3.

In some embodiments, said functional variant has at least 90% sequence identity to said hGIP (3-30) as set forth in SEQ ID NO: 3.

In some embodiments, said functional variant has at least 95% sequence identity to said hGIP (3-30) as set forth in SEQ ID NO: 3.

In some embodiments, said GIP activity modulator is a GIP peptide analogue of sequence hGIP (3-30) as set forth in SEQ ID NO: 3, or a functional variant thereof having 1 to 8 individual amino acid substitutions, such as 1 to 7 individual amino acid substitutions, such as 1 to 6 individual amino acid substitutions, such as 1 to 5 individual amino acid substitutions, such as 1 to 4 individual amino acid substitutions, such as 1 to 3 individual amino acid substitutions, such as 1 to 2 individual amino acid substitutions.

In some embodiments, said GIP activity modulator is a GIP peptide analogue of sequence hGIP (3-30) as set forth in SEQ ID NO: 3, or a functional variant thereof having 1 amino acid substitution; such as a peptide analogue of sequence hGIP (3-30) as set forth in SEQ ID NO: 3, or a functional variant thereof having 2 individual amino acid substitutions; such as a peptide analogue of sequence hGIP (3-30) as set forth in SEQ ID NO: 3, or a functional variant thereof having 3 individual amino acid substitutions; such as a peptide analogue of sequence hGIP (3-30) as set forth in SEQ ID NO: 3, or a functional variant thereof having 4 individual amino acid substitutions; such as a peptide analogue of sequence hGIP (3-30) as set forth in SEQ ID NO: 3, or a functional variant thereof having 5 individual amino acid substitutions; such as a peptide analogue of sequence hGIP (3-30) as set forth in SEQ ID NO: 3, or a functional variant thereof having 6 individual amino acid substitutions, or a functional variant thereof having 7 individual amino acid substitutions; such as a peptide analogue of sequence hGIP (3-30) as set forth in SEQ ID NO: 3, or a functional variant thereof having 8 individual amino acid substitutions; such as a peptide analogue of sequence hGIP (3-30) as set forth in SEQ ID NO: 3.

In one embodiment, the GIP activity modulator comprises a GIP peptide analogue of sequence hGIP (3-30) as set forth in SEQ ID NO: 3, or a functional variant thereof capable of binding to and antagonising a GIPR.

In some embodiments, the GIP activity modulator for use according to the present disclosure is a GIP peptide analogue which is a functional fragment of hGIP (3-30) as set forth in SEQ ID NO:3, wherein said functional fragment comprises 20 to 28 amino acids of said GIP (3-30); and is capable of binding a GIPR.

In some embodiments, the functional fragment of GIP (3-30) as set forth in SEQ ID NO: 3 comprises 22 to 28 amino acids of GIP (3-30), such as 22 to 26 amino acids, such as 22 to 24 amino acids, such as 24 to 28 amino acids, such as 24 to 26 amino acids, such as 26 to 28 amino acids of GIP (3-30).

In one embodiment, the GIP activity modulator comprises a GIP peptide analogue of sequence hGIP (5-30) as set forth in SEQ ID NO:7, or a functional variant thereof capable of binding to and antagonising a GIPR.

In some embodiments, the GIP activity modulator for use according to the present disclosure is a GIP peptide analogue which is a functional fragment of hGIP (5-30) as set forth in SEQ ID NO:7, wherein said functional fragment comprises 20 to 26 amino acids of said GIP (3-30); and is capable of binding a GIPR.

In some embodiments, the functional fragment of GIP (5-30) as set forth in SEQ ID NO: 3 comprises 22 to 25 amino acids of GIP (3-30), such as 22 to 24 amino acids, such as 24 to 26 amino acids, such as 23 to 25 amino acids of GIP (5-30).

In some embodiments, said GIP activity modulator comprises a GIP peptide analogue selected from hGIP (3-42) as set forth in SEQ ID NO: 2, hGIP (4-42) as set forth in SEQ ID NO: 4 and hGIP (5-42) as set forth in SEQ ID NO: 5, or a functional variant thereof having at least 75% sequence identity to any one of hGIP (3-42) as set forth in SEQ ID NO: 2, hGIP (4-42) as set forth in SEQ ID NO: 4 and hGIP (5-42) as set forth in SEQ ID NO: 5.

In some embodiments, said GIP activity modulator comprises a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having at least 70% sequence identity to said peptide.

In some embodiments, said GIP activity modulator comprises a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 1 to 8 amino acid substitutions, such as 1 to 7 amino acid substitutions, such as 1 to 6 amino acid substitutions, such as 1 to 5 amino acid substitutions, such as 1 to 4 amino acid substitutions, such as 1 to 3 amino acid substitutions, such as 1 to 2 amino acid substitutions, such as 1, 2, 3, 4, 5, 6, 7 or 8 individual amino acid substitutions of any one of SEQ NOs: 3 and 6-9.

In some embodiments, said GIP activity modulator comprises a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 1 amino acid substitution; such as a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 2 individual amino acid substitutions; such as a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 3 individual amino acid substitutions; such as a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 4 individual amino acid substitutions; such as a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 5 individual amino acid substitutions; such as a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 6 individual amino acid substitutions, such as a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 7 individual amino acid substitutions, such as a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant thereof having 8 individual amino acid substitutions.

In one embodiment a functional variant of the peptide sequence of hGIP (3-30) as set forth in SEQ ID NO: 3, retains the same biological activities or capabilities as the native peptide or the peptide from which it is derived. In one embodiment a functional variant of the peptide sequence of hGIP (5-30) as set forth in SEQ ID NO: 7, retains the same biological activities or capabilities as the native peptide or the peptide from which it is derived. In one embodiment a GIP peptide analogue and a functional variant thereof or a functional fragment thereof as defined herein is capable of one or more of: Binding to one or more GIPR; antagonizing one or more GIPR; displacing GIP (1-42) as set forth in SEQ ID NO: 1 and/or GIP (1-30) as set forth in SEQ ID NO: 3 from one or more GIPR; having a higher, equal or lower affinity for a given GIPR than GIP (1-42) as set forth in SEQ ID NO: 1 and/or GIP (1-30) as set forth in SEQ ID NO: 3; antagonizing somatostatin secretion induced by native GIP, GIP (1-42) as set forth in SEQ ID NO: 1 and/or GIP (1-30) as set forth in SEQ ID NO: 3; antagonizing insulin secretion induced by native GIP, GIP (1-42) as set forth in SEQ ID NO: 1 and/or GIP (1-30) as set forth in SEQ ID NO: 3; and antagonising glucagon secretion induced by native GIP, GIP (1-42) as set forth in SEQ ID NO: 1 and/or GIP (1-30) as set forth in SEQ ID NO: 3.

In one embodiment a GIP peptide analogue and a functional variant thereof and a functional fragment thereof as defined herein is capable of binding (or binds) to one or more of the hGIPR (Uniprot accession number P48546), the rGIPR (Uniprot accession number P43219), the mGIPR (Uniprot accession number QOP543), the dog GIPR (Uniprot accession number E2RIK5), the pig GIPR (Uniprot accession number I3LND8), and the Macaca mulatta GIPR (Uniprot accession number A0A1D5QDM0) (primate).

### C-Terminal Elongation

In some embodiments, said GIP activity modulator comprises a GIP peptide analogue as disclosed herein. In some embodiments, said GIP activity modulator is a GIP peptide analogue as disclosed herein which is elongated, such as C-terminally elongated, such as elongated by addition of one or more C-terminal amino acid residues, such as a C-terminal peptide.

In some embodiments, said GIP peptide analogue as disclosed herein is C-terminally elongated by one or more amino acid residues, such as a C-terminal additional peptide.

In one embodiment, the GIP activity modulator comprises a peptide analogue of sequence hGIP (3-30) as set forth in SEQ ID NO: 3, or a functional fragment or variant thereof, wherein said functional variant has at least 70% sequence identity SEQ ID NO: 3, such as wherein said variant has 1 to 8 individual amino acid substitutions compared to SEQ ID NO: 3, and comprising one or more C-terminal amino acid residues, such as comprising a C-terminal peptide.

In one embodiment, the GIP activity modulator comprises a peptide analogue of sequence hGIP (5-30) as set forth in SEQ ID NO: 7, or a functional fragment or variant thereof, wherein said functional variant has at least 70% sequence identity SEQ ID NO:

7, such as wherein said variant has 1 to 8 individual amino acid substitutions compared to SEQ ID NO: 7, and comprising one or more C-terminal amino acid residues, such as comprising a C-terminal peptide.

In one embodiment, the GIP activity modulator comprises a peptide analogue of sequence hGIP (5-31) as set forth in SEQ ID NO: 16, or a functional fragment or variant thereof, wherein said functional variant has at least 70% sequence identity SEQ ID NO: 16, such as wherein said variant has 1 to 8 individual amino acid substitutions compared to SEQ ID NO: 16, and comprising one or more C-terminal amino acid residues, such as comprising a C-terminal peptide.

In some embodiments, said C-terminal peptide comprises one or more amino acid residues of GIP (31-42) (GKKNDWKHNITQ; SEQ ID NO: 10).

In some embodiments, said C-terminal peptide comprises one or more amino acid residues of Exendin-4 (HGEGTFTSDLSKQMEEEAVRLFIEWLKNGGPSSGAPPPS; SEQ ID NO: 11).

### Fatty Acids

In some embodiments, said GIP activity modulator is a GIP peptide analogue as disclosed herein which is modified by attaching at least one fatty acid molecule at one or more amino acid residues of said peptide.

In some embodiments, said GIP activity modulator is a GIP peptide analogue as disclosed herein which is modified by attaching at least one fatty acid molecule at one or more amino acid residues of said GIP peptide analogue, or a functional variant thereof, wherein said GIP peptide analogue may be conjugated to an additional peptide and wherein said GIP peptide analogue may be C-terminally amidated.

In some embodiments, said at least one fatty acid molecule is a straight-chain fatty acid.

In some embodiments, said at least one fatty acid molecule is a branched fatty acid.

In some embodiments, said at least one fatty acid molecule is a monoacyl fatty acid molecule, comprising one fatty acid.

In some embodiments, said at least one fatty acid molecule is a diacyl fatty acid molecule.

In some embodiments, at least one of said at least one fatty acid molecule is attached to an amino acid residue directly.

In some embodiments, at least one of said at least one fatty acid molecule is attached to an amino acid residue via a linker.

In one embodiment, the GIP activity modulator comprises a peptide analogue of sequence hGIP (3-30) as set forth in SEQ ID NO: 3, or a functional fragment or variant thereof, wherein said functional variant has at least 70% sequence identity SEQ ID NO: 3, such as wherein said variant has 1 to 8 individual amino acid substitutions compared to SEQ ID NO: 3; optionally comprising one or more C-terminal amino acid residues, such as comprising a C-terminal peptide; wherein said peptide analogue comprises at least one fatty acid molecule.

In one embodiment, the GIP activity modulator comprises a peptide analogue of sequence hGIP (5-30) as set forth in SEQ ID NO: 7, or a functional fragment or variant thereof, wherein said functional variant has at least 70% sequence identity SEQ ID NO: 7, such as wherein said variant has 1 to 8 individual amino acid substitutions compared to SEQ ID NO: 7; optionally comprising one or more C-terminal amino acid residues, such as comprising a C-terminal peptide; wherein said peptide analogue comprises at least one fatty acid molecule.

In one embodiment, the GIP activity modulator comprises a peptide analogue of sequence hGIP (5-31) as set forth in SEQ ID NO: 16, or a functional fragment or variant thereof, wherein said functional variant has at least 70% sequence identity SEQ ID NO: 16, such as wherein said variant has 1 to 8 individual amino acid substitutions compared to SEQ ID NO: 16; optionally comprising one or more C-terminal amino acid residues, such as comprising a C-terminal peptide; wherein said peptide analogue comprises at least one fatty acid molecule.

### Function of Compound

In some embodiments, the GIP activity modulator of the present disclosure reduces the heart rate of an individual during and/or after an orthostatic challenge. In some embodiments, the GIP activity modulator of the present disclosure reduces the fasting heart rate of an individual during and/or after an orthostatic challenge. In some embodiments, the GIP activity modulator of the present disclosure reduces or prevents postprandial increase of heart rate in an individual during and/or after an orthostatic challenge.

An orthostatic challenge occurs when an individual moves from a supine position to a standing position, or from a supine position to a sitting position. An orthostatic challenge may also occur in an individual tilting up their head of at least 60°.

In some embodiments, the GIP activity modulator of the present disclosure reduces or prevents tachycardia in an individual during and/or after an orthostatic challenge. In some embodiments, the GIP activity modulator of the present disclosure reduces or prevents fasting tachycardia in an individual during and/or after an orthostatic challenge. In some embodiments, the GIP activity modulator of the present disclosure reduces or prevents postprandial tachycardia in an individual during and/or after an orthostatic challenge.

In some embodiments, the GIP activity modulator of the present disclosure increases the blood pressure of an individual during and/or after an orthostatic challenge. In some embodiments, the GIP activity modulator of the present disclosure increases the systolic blood pressure of an individual during and/or after an orthostatic challenge. In some embodiments the GIP activity modulator of the present disclosure increases the diastolic blood pressure of an individual during and/or after an orthostatic challenge.

In some embodiments, the GIP activity modulator of the present disclosure increases the fasting blood pressure of an individual during and/or after an orthostatic challenge, such as increases the fasting systolic blood pressure and/or increases the fasting diastolic blood pressure during and/or after an orthostatic challenge.

In some embodiments, the GIP activity modulator of the present disclosure prevents a decrease in the blood pressure of an individual during and/or after an orthostatic challenge. In some embodiments, the GIP activity modulator of the present disclosure prevents a decrease in the systolic blood pressure of an individual during and/or after an orthostatic challenge. In some embodiments the GIP activity modulator of the present disclosure prevents a decrease in the diastolic blood pressure of an individual during and/or after an orthostatic challenge.

In some embodiments, the GIP activity modulator of the present disclosure prevents a decrease in the fasting blood pressure of an individual during and/or after an orthostatic challenge, such as prevents a decrease in the fasting systolic blood pressure and/or the fasting diastolic blood pressure during and/or after an orthostatic challenge.

In some embodiments, the GIP activity modulator of the present disclosure increases the postprandial blood pressure of an individual during and/or after an orthostatic challenge, such as increases the postprandial systolic blood pressure and/or increases the postprandial diastolic blood pressure during and/or after an orthostatic challenge.

In some embodiments, the GIP activity modulator of the present disclosure reduces the blood flow in the superior mesenteric artery such as reduces the fasting blood flow in the superior mesenteric artery of an individual. In some embodiments, the GIP activity modulator of the present disclosure reduces the blood flow in the superior mesenteric artery such as the fasting blood flow in the superior mesenteric artery of an individual during and/or after an orthostatic challenge.

In some embodiments, the GIP activity modulator of the present disclosure inhibits GIPR activity at least about 80%, such as at least about 85%, such as at least about 90%, such as at least about 95%, such as about 100%; such as wherein said inhibition of GIPR activity is determined as a decrease in intracellular cAMP.

In some embodiments, the GIP activity modulator of the present disclosure inhibits GIP activity, such as inhibits GIP activity at least about 50%, such as at least about 60%, such as at least about 70%, such as at least about 80%, such as at least about 85%, such as at least about 90%, such as at least about 95%, such as about 100%.

In some embodiments, the GIP activity modulator of the present disclosure is capable of one or more of the following:
a. binding to a GIPR,
b. activating a GIPR,
c. antagonizing a GIPR,
d. inhibiting or reducing GIPR downstream signalling, such as cAMP generation,
e. decreasing intracellular cAMP,
f. disrupting the interaction between GIP and GIPR,
g. increasing or decreasing GIP secretion,
h. inhibiting or reducing agonist-mediated responses of GIPR, and/or
i. inhibiting GIPR activity at least 80%, such as at least 85%, such as at least 90%, such as at least 95%, such as about 100%.

In some embodiments the GIP activity modulator of the present disclosure is capable of inhibiting (reducing, decreasing, antagonizing) one or more of i) GIP-induced glucagon secretion, ii) GIP-induced insulin secretion, iii) GIP-induced somatostatin secretion, iv) GIP-induced glucose uptake, v) GIP-induced fatty acid synthesis and/or fatty acid incorporation, vi) high or increased expression or activity of a GIPR and vii) release of GIP following a meal (post-prandial GIP release).

### Medical Use

It is an aspect of the present disclosure to provide a GIP activity modulator as disclosed herein for use in the treatment of orthostatic intolerance.

In some embodiments, said orthostatic intolerance is orthostatic intolerance with orthostatic hypotension and/or orthostatic intolerance with orthostatic tachycardia.

It is an aspect of the present disclosure to provide a GIP activity modulator as disclosed herein for use in the treatment of postprandial orthostatic intolerance.

Orthostatic intolerance may be defined as “the development of symptoms while upright, during standing that are relieved by recumbency”.

It is also an aspect of the present disclosure to provide the use of a GIP activity modulator as disclosed herein for the manufacture of a medicament for the treatment of orthostatic intolerance.

Another aspect is to provide a method of treating orthostatic intolerance, the method comprising one or more steps of administering a therapeutically effective amount of a GIP activity modulator according to the present disclosure to an individual in need thereof.

Also disclosed in a method of treating orthostatic hypotension, such as postprandial orthostatic hypotension, the method comprising one or more steps of administering a therapeutically effective amount of a GIP activity modulator according to the present disclosure to an individual in need thereof.

In some embodiments said orthostatic intolerance is orthostatic intolerance with hypermobility spectrum disorders/hypermobile Ehlers-Danlos syndrome (HSD/hEDS).

Also disclosed in a method of treating orthostatic hypotension selected from the group consisting of initial orthostatic hypotension, classical orthostatic hypotension, delayed orthostatic hypotension, neurogenic hypotension and POTS, the method comprising one or more steps of administering a therapeutically effective amount of a GIP activity modulator according to the present disclosure to an individual in need thereof.

Also disclosed in a method of treating reduction of blood pressure, such as postprandial reduction of blood pressure, the method comprising one or more steps of administering a therapeutically effective amount of a GIP activity modulator according to the present disclosure to an individual in need thereof.

Also disclosed in a method of treating orthostatic tachycardia, such as postprandial orthostatic tachycardia, the method comprising one or more steps of administering a therapeutically effective amount of a GIP activity modulator according to the present disclosure to an individual in need thereof.

Also disclosed in a method of treating orthostatic increase in heart rate, such as postprandial orthostatic increase in heart rate, the method comprising one or more steps of administering a therapeutically effective amount of a GIP activity modulator according to the present disclosure to an individual in need thereof.

Also disclosed in a method of treating orthostatic syncope, orthostatic presyncope, orthostatic dizziness and orthostatic light-headedness, the method comprising one or more steps of administering a therapeutically effective amount of a GIP activity modulator according to the present disclosure to an individual in need thereof.

Also disclosed in a method of treating postprandial orthostatic syncope, postprandial orthostatic presyncope, postprandial orthostatic dizziness and postprandial orthostatic light-headedness, heart palpitations, problems with thinking, memory and concentration, the method comprising one or more steps of administering a therapeutically effective amount of a GIP activity modulator according to the present disclosure to an individual in need thereof.

It is also an aspect of the present disclosure to provide a method of reducing heart rate and/or preventing an increase in heart rate, in an individual with orthostatic challenges, such as an individual with orthostatic intolerance, said method comprising one or more steps of administering to said individual a therapeutically effective amount of a GIP activity modulator according to the present disclosure.

An individual with orthostatic challenges is an individual that moves from a supine position to a standing position, an individual who moves from a supine position to a sitting position; or an individual tilting up their head at least 60°.

In one embodiment an individual with orthostatic challenges is an individual with POTS, or an individual diagnosed with POTS.

In one embodiment an individual with orthostatic intolerance is an individual with POTS, or an individual diagnosed with POTS.

It is also an aspect of the present disclosure to provide a method of increasing blood pressure and/or preventing a reduction in blood pressure,
such as increasing diastolic blood pressure and/or preventing a reduction in diastolic blood pressure, and/or
and/or increasing systolic blood pressure and/or preventing a reduction in systolic blood pressure,
in an individual with orthostatic challenges, such as an individual with orthostatic intolerance, said method comprising one or more steps of administering to said individual a therapeutically effective amount of a GIP activity modulator according to the present disclosure.

It is also an aspect of the present disclosure to provide a method of reducing or preventing syncope in an individual with orthostatic challenges, such as an individual with orthostatic intolerance, said method comprising one or more steps of administering to said individual a therapeutically effective amount of a GIP activity modulator according to the present disclosure.

It is also an aspect of the present disclosure to provide a method of reducing or preventing presyncope in an individual with orthostatic challenges, such as an individual with orthostatic intolerance, said method comprising one or more steps of administering to said individual a therapeutically effective amount of a GIP activity modulator according to the present disclosure.

It is also an aspect of the present disclosure to provide a method of reducing or preventing dizziness in an individual with orthostatic challenges, such as an individual with orthostatic intolerance, said method comprising one or more steps of administering to said individual a therapeutically effective amount of a GIP activity modulator according to the present disclosure.

It is also an aspect of the present disclosure to provide a method of reducing or preventing light-headedness in an individual with orthostatic challenges, such as an individual with orthostatic intolerance, said method comprising one or more steps of administering to said individual a therapeutically effective amount of a GIP activity modulator according to the present disclosure.

In some embodiments of the present disclosure, said orthostatic intolerance is orthostatic hypotension.

In some embodiments of the present disclosure, said orthostatic hypotension is selected from the group consisting of initial orthostatic hypotension, classical orthostatic hypotension and delayed orthostatic hypotension.

In some embodiments of the present disclosure, the individual in need thereof has POTS, or is diagnosed with POTS.

In some embodiments of the present disclosure, said orthostatic intolerance is POTS.

In some embodiments the individual with orthostatic intolerance or orthostatic hypotension has a sustained reduction of systolic BP of at least 20 mmHg; a sustained reduction of diastolic BP of at least 10 mmHg, and/or a sustained decrease in systolic BP to an absolute value 90 mmHg or lower after standing, or after a head-up tilt of at least 60 degrees.

Classical orthostatic hypotension is defined as a sustained decrease in systolic blood pressure (BP) ≥20 mmHg, a sustained decrease in diastolic BP ≥10 mmHg, and/or a sustained decrease in systolic BP to an absolute value <90 mmHg within 3 min of active standing or head-up tilt of at least 60 degrees. In cases of an individual with existing hypertension, a systolic BP decrease ≥30 mmHg should be considered.

Initial OH is characterized by a BP decrease on standing of >40 mmHg for systolic BP and/or >20 mmHg for diastolic BP within 15 s of standing. BP then spontaneously and rapidly returns to normal, so the period of hypotension and symptoms is short (<40 s) but may still cause syncope.

Delayed OH is defined as OH occurring beyond 3 min of head-up tilt or active standing.

Thus, in some embodiments of the present disclosure, the individual with orthostatic intolerance and/or orthostatic hypotension has a sustained reduction of systolic BP of at least 20 mmHg, such as of at least 22 mmHg, such as of at least 25 mmHg, such as of at least 28 mmHg, such as of at least 30 mmHg within 3 minutes of standing, or sitting up, or head-up tilt of at least 60 degrees.

In some embodiments of the present disclosure, the individual with orthostatic intolerance and/or orthostatic hypotension has a sustained reduction of diastolic BP of at least 10 mmHg, such as of at least 12 mmHg, such as of at least 15 mmHg, such as of at least 18 mmHg, such as of at least 20 mmHg within 3 minutes of standing, or sitting up, or head-up tilt of at least 60 degrees.

In some embodiments of the present disclosure, the individual with orthostatic intolerance and/or orthostatic hypotension has a sustained decrease in systolic BP to an absolute value 90 mmHg or lower, such as of 88 mmHg or lower, such as of 85 mmHg or lower, such as of 83 mmHg or lower, such as of 81 mmHg or lower within 3 minutes of standing, or sitting up, or head-up tilt of at least 60 degrees.

Similarly, in some embodiments of the present disclosure, the individual with orthostatic intolerance has an increase in heart rate of more than 30 bpm, such as of more than 33 bpm, more than 35 bpm, more than 38 bpm, more than 40 bpm within 10 minutes of standing, or sitting up, or head-up tilt of at least 60 degrees.

In some embodiments of the present disclosure, the individual with orthostatic intolerance has a heart rate of at least 120 bpm, such as of at least 122 bpm, such as of at least 125 bpm, such as of at least 130 bpm, such as of at least 135 bpm within 10 minutes of standing, or sitting up, or head-up tilt of at least 60 degrees.

In some embodiments of the present disclosure, the individual with orthostatic challenges is a female individual.

In some embodiments of the present disclosure, the individual with orthostatic intolerance is a female individual.

In some embodiments of the present disclosure, the individual with orthostatic hypotension is a female individual.

In some embodiments of the present disclosure, female individual is a female human being.

The present disclosure further relates to a method for treatment of POTS comprising administration of a GIP activity modulator as disclosed herein to an individual in need thereof.

In some aspects, the present disclosure relates to a pharmaceutical composition, such as pharmaceutically acceptable composition, comprising a GIP activity modulator as disclosed herein and a pharmaceutically acceptable carrier, for use in the treatment of orthostatic intolerance.

Postural tachycardia syndrome is a type of Ol that is prevalent in young women. POTS is a debilitating syndrome. The symptoms of POTS include, but are not limited to: dizziness or light headedness, fainting, problems with thinking, memory and concentration, heart palpitations, shaking and sweating, weakness and fatigue (tiredness), headaches, poor sleep, chest pain, feeling sick, and shortness of breath. POTS is also characterized by a marked orthostatic HR increase (>30 bpm, or >120 bpm within 10 min of standing or head-up tilt in the absence of OH). In some embodiments, the GIP activity modulator of the present disclosure reduces or ameliorates one or more of the symptoms of POTS.

Also disclosed is a method of treating orthostatic intolerance, the method comprising one or more steps of administering a GIP activity modulator, or a pharmaceutical composition comprising a GIP activity modulator, and further comprising one or more steps of administering one or more additional therapeutic agents for use in the treatment of orthostatic intolerance.

### Pharmaceutical Composition and Formulation

Whilst it is possible for the bioactive agent of the present disclosure to be administered as the raw chemical, such as a peptide, it is sometimes preferred to present them in the form of a pharmaceutical formulation. Such a pharmaceutical formulation may be referred to as a pharmaceutical composition, pharmaceutically acceptable composition or pharmaceutically safe composition.

Accordingly, further provided is a pharmaceutical formulation, which comprises a bioactive agent of the present disclosure, or a pharmaceutically acceptable salt or ester thereof, and a pharmaceutically acceptable carrier, excipient and/or diluent. The pharmaceutical formulations may be prepared by conventional techniques, e.g. as described in Remington: The Science and Practice of Pharmacy 2005, Lippincott, Williams & Wilkins.

Pharmaceutically acceptable salts of the instant compounds, where they can be prepared, are also intended to be covered by this disclosure. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds as disclosed herein may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

### Routes of Administration

It will be appreciated that the preferred route of administration will depend on the general condition and age of the subject to be treated, the nature of the condition to be treated, the location of the tissue to be treated in the body and the active ingredient chosen.

### Systemic Treatment

For systemic treatment according to the present disclosure the route of administration is capable of introducing the bioactive agent into the blood stream to ultimately target the sites of desired action.

Such routes of administration are any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intracerebral, intravenous and intradermal administration).

### Local Treatment

The bioactive agent according to the invention may in one embodiment be used as a local treatment, i.e. be introduced directly to the site(s) of action.

### EXAMPLES

### Example 1

### Methods

### Test Substance and Subcutaneous Injections

Synthetic human GIP (1-42) (GIP) (PolyPeptide Group, Strasbourg, France) was dissolved in a sodium hydrogen carbonate buffer with 0.5% human serum albumin in NaCl 9 mg/ml to a final concentration of 4 mg/ml. The solution was sterile filtered and dispensed into glass ampoules. On the appropriate study days, the GIP solutions were further diluted in saline (NaCl 9 mg/ml) to 125 μg/ml. Saline (NaCl 9 mg/mL) was used as placebo.

At the beginning of each study day, participants were weighed and received either a placebo injection or a weight-dependent GIP (1-42) dose of 2.7 μg/kg at time 0.

### Participants and Procedures

Four healthy, non-medicated participants between 33-40 years old (one male and three females) were included in the study. On each of the two study days, participants arrived after an overnight fast (at least 10 h fasting). To enable continuous heart rate monitoring, three electrodes compatible with the Holter Spiderview recorder were placed over the rib at V5, on the xiphoid muscle at the bottom of the sternum, and at the sternum over the manubrium after the skin was disinfected or shaved and disinfected for the male. Baseline characterization was initiated at timepoint-60 min and lasted for one hour. For baseline characterization, the participants alternated between the supine position and 10 min orthostatic challenges initiated at timepoints −45 min and −20 min. Orthostatic challenges were elicited by actively standing up from the hospital bed. At timepoint 0 min, the participants received a subcutaneous injection of either 2.7 μg/kg GIP or saline in a single-blinded manner. Following the injection, the participants rested in the supine position for one hour when not doing 10 min orthostatic challenges initiated at 15 min and 40 min post-injection. At time points −50 min, −40 min, −30 min, 10 min, 20 min, 45 min, and 55 min systolic- and diastolic blood pressure as well as heart rate were measured using the Omron M2 blood pressure monitor. The two study days were identical except for the injection of either GIP or saline. Fasting prior to administration of GIP mimics the body's natural response to eating, as eating induces GIP secretion. The method employed thus mimics fasting or postprandial conditions with the emphasis on GIP's physiological effects.

### Results

### Heart Rate

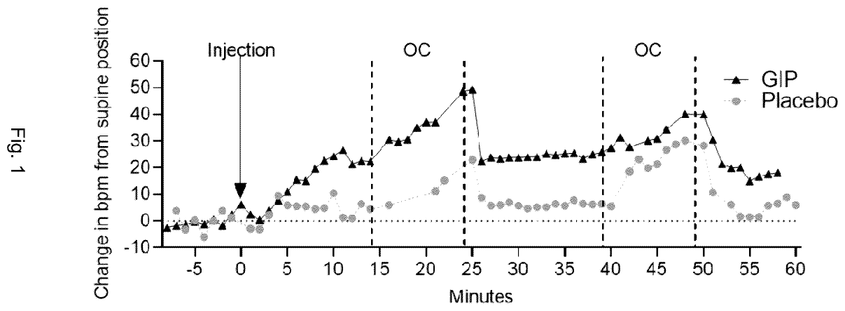

The results are shown in FIG. 1. During baseline characterization, there was no change in heart rate when in the supine position and there was an increase in heart rate of 12-15 beats per minute (bpm) during the orthostatic challenge. Five min after the orthostatic challenge, the heart rate decreased to pre-challenge supine levels. The GIP infusion caused a substantial increase in heart rates, both in the supine position and during the orthostatic challenge (17 vs. 36 bpm over baseline supine levels, respectively). The placebo infusion did not evoke any differences in heart rates compared to the baseline characterization (0 vs. 16 bpm for the supine position and during the orthostatic challenge, respectively).

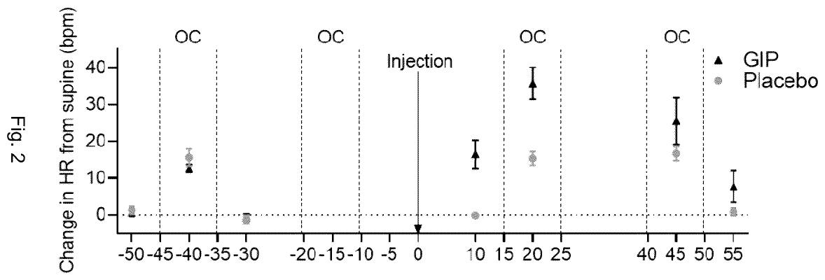

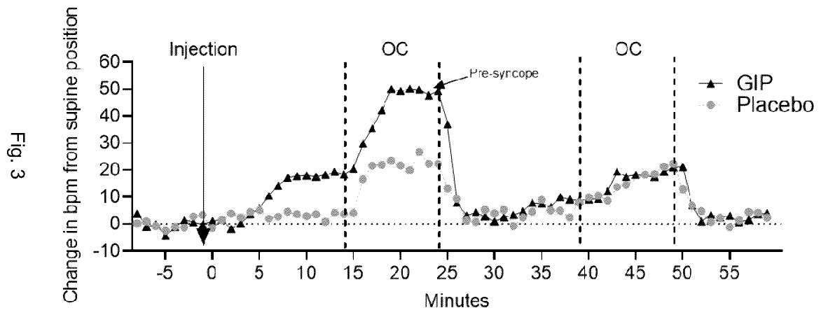

As the study participants were blinded, this demonstrates a negligible placebo effect. Due to sub-optimal positioning of the electrodes in two participants, resulting in poor data quality, a more granular (using the Holter monitor) assessment of changes in heart rate is only available for two of the study participants. The results are shown in FIGS. 2 and 3. Compared to baseline supine heart rate, the GIP injection mediated a gradual increase in supine heart rate of 19-28 bpm within 15 min depending on the subject. The GIP-injection mediated an increase of 49-50 bpm compared to baseline supine heart rate during the first orthostatic challenge post injection.

### Blood Pressure

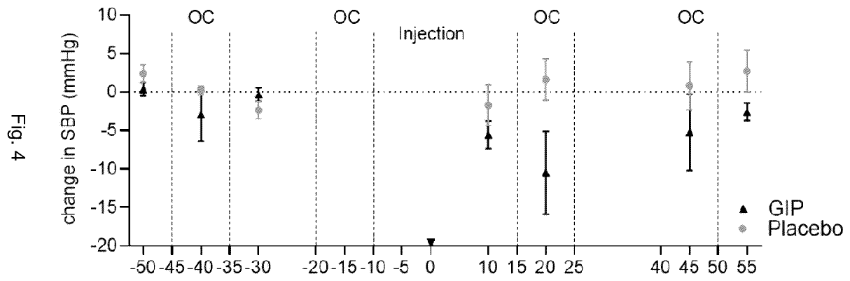

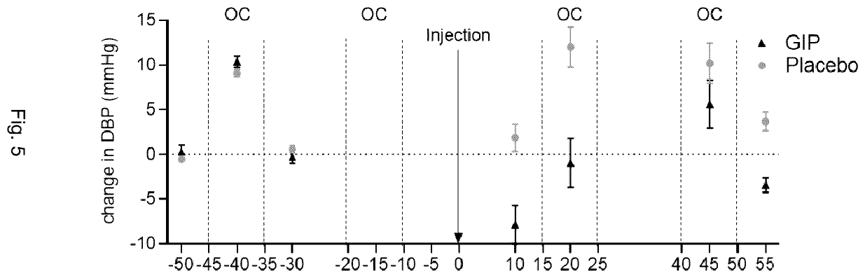

The results are shown in FIGS. 4 and 5. During baseline and after the placebo injection, systolic blood pressure (SBP) was stable in both the supine position and during the orthostatic challenge. The GIP-infusion mediated a decrease in SBP of 12 mmHg during the orthostatic challenges and a decrease of 4-5 mmHg while in the supine position compared to placebo levels.

Diastolic blood pressure (DBP) increased both during the orthostatic challenge and in the supine position on the placebo day (10-12 mmHg and 2 mmHg from baseline supine levels, respectively). After GIP injection, DBP decreased both during the orthostatic challenge and in the supine position (−13 mmHg and −10 mmHg compared to placebo levels, respectively).

### GIP and Clinical Signs

The dose of GIP was similar to that of previous studies targeting postprandial physiological plasma concentrations. However, as the half-life of GIP is ~4 min, the effects of GIP in this experimental setup are expected to be more transient when compared to the sustained GIP secretion (lasting hours) during normal postprandial physiological circumstances.

During the first orthostatic challenge following GIP administration, one of the subjects experienced presyncope and had to lay down. Three out of four subjects reported significant dizziness and lightheadedness during the orthostatic challenges post GIP-injection. No clinical signs were reported following placebo administration.

### CONCLUSION

Taken together, these results demonstrate for the first time that GIP significantly worsens orthostatic tolerance by decreasing blood pressure (both SBP and DBP) as well as by increasing heart rate in both men and women.

Consequently, these data support the involvement of GIP in postural haemodynamic changes and support the notion of GIP activity modulation as an innovative therapeutic approach to treat autonomic diseases associated with orthostatic intolerance including orthostatic hypotension and orthostatic increases in heart rate including tachycardia.

Sequence overview
SEQ ID NO: 1 - native hGIP1-42:
YAEGTFISDYSIAMDKIHQQDFVNWLLAQKGKKNDWKHNITQ
SEQ ID NO: 2 - hGIP3-42:
EGTFISDYSIAMDKIHQQDFVNWLLAQKGKKNDWKHNITQ
SEQ ID NO: 3 - hGIP3-30:
EGTFISDYSIAMDKIHQQDFVNWLLAQK
SEQ ID NO: 4 - hGIP4-42:
GTFISDYSIAMDKIHQQDFVNWLLAQKGKKNDWKHNITQ
SEQ ID NO: 5 - hGIP5-42:
TFISDYSIAMDKIHQQDFVNWLLAQKGKKNDWKHNITQ
SEQ ID NO: 6 - hGIP4-30:
GTFISDYSIAMDKIHQQDFVNWLLAQK
SEQ ID NO: 7 - hGIP5-30:
TFISDYSIAMDKIHQQDFVNWLLAQK
SEQ ID NO: 8 - hGIP6-30:
FISDYSIAMDKIHQQDFVNWLLAQK
SEQ ID NO: 9 - hGIP7-30:
ISDYSIAMDKIHQQDFVNWLLAQK
SEQ ID NO: 10 - hGIP(31-42):
GKKNDWKHNITQ
SEQ ID NO: 11 - Exendin-4:
HGEGTFTSDLSKQMEEEAVRLFIEWLKNGGPSSGAPPPS
SEQ ID NO: 12:
QVQLVESGGGVVQPGRSLRLSCAASGFTFSNYGMHWVRQAPGEGL
EWVAAIWFDASDKYYADAVKGRFTISRDNSKNTLYLQMNSLRAED
TAVYYCARDQAIFGVVPDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSC
SEQ ID NO: 13:
EIVMTQSPATLSVSPGERATLSCRASQSVSSNLAWYQQKPGQAPR
LLIYGAATRATGIPARVSGSGSGTEFTLTISSLQSEDFAVYYCQQ
YNNWPLTFGGGTKVEIKRTVAAPSVFIFPPSDEQLKSGTASWVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 14:
QVQLQQSGAEVKKPGSSVKVSCKASGGTFSSYAISWVRQAPGQGL
EWMGGIIPTFGTANYAQKFQGRVTITADESTSTAYMELSSLRSED
TAVYYCAQGPIVGAPTDYWGKGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKRVEPKSCDKT
HT
SEQ ID NO: 15:
SYVLTQPPSASGTPGQRVAISCSGSNSNIGSNTVHWYQQLPGAAP
KLLIYSNNQRPSGVPDRFSGSNSGTSASLAISRLQSEDEADYYCA
AWDDSLNGVVFGGGTKVTVLQPKAAPSVTLFPPSSEELQANKATL
VCLISDFYPGAVTVAWKADSSPVKAGVETTTPSKQSNNKYAASSY
LSLTPEQWKSHRSYSCQVTHEGSTVEKTVAPTECS
SEQ ID NO: 16: hGIP5-31:
TFISDYSIAMDKIHQQDFVNWLLAQKG

1. A method of treating orthostatic intolerance, the method comprising one or more steps of administering a therapeutically effective amount of a GIP activity modulator to an individual in need thereof.

2. The method according to claim 1, wherein said orthostatic intolerance is orthostatic intolerance with orthostatic hypotension and/or orthostatic intolerance with orthostatic tachycardia.

3. The method according to claim 1, wherein said orthostatic intolerance is orthostatic hypotension, such as postprandial orthostatic hypotension.

4. The method according to claim 1, wherein said orthostatic intolerance is reduction of blood pressure, such as postprandial reduction of blood pressure.

5. The method according to claim 1, wherein said orthostatic intolerance is orthostatic tachycardia, such as postprandial orthostatic tachycardia.

6. The method according to claim 1, wherein said orthostatic intolerance is orthostatic increase in heart rate, such as postprandial orthostatic increase in heart rate.

7. The method according to claim 1, wherein said orthostatic intolerance is selected from the group consisting of orthostatic syncope, orthostatic presyncope, orthostatic dizziness and orthostatic light-headedness.

8. The method according to claim 1, wherein said orthostatic intolerance is selected from the group consisting of postprandial orthostatic syncope, postprandial orthostatic presyncope, postprandial orthostatic dizziness and postprandial orthostatic light-headedness.

9. The method according to claim 1, wherein said orthostatic intolerance is orthostatic intolerance with hypermobility spectrum disorders/hypermobile Ehlers-Danlos syndrome (HSD/hEDS).

10. A method of reducing heart rate and/or preventing an increase in heart rate, in an individual with orthostatic challenges, such as an individual with orthostatic intolerance, said method comprising one or more steps of administering to said individual a therapeutically effective amount of a GIP activity modulator.

11. A method of increasing blood pressure and/or preventing a reduction in blood pressure, such as in diastolic and/or in systolic blood pressure, in an individual with orthostatic challenges, such as an individual with orthostatic intolerance, said method comprising one or more steps of administering to said individual a therapeutically effective amount of a GIP activity modulator.

12. A method of reducing or preventing one or more of syncope, presyncope, dizziness or light-headedness, heart palpitations, problems with thinking, memory and concentration, in an individual with orthostatic challenges, such as an individual with orthostatic intolerance, said method comprising one or more steps of administering to said individual a therapeutically effective amount of a GIP activity modulator.

13. The method according to any one of the preceding claims, wherein said individual with orthostatic challenges is an individual moving from a supine position to a standing position; or an individual moving from a supine position to a sitting position; or an individual tilting up their head at least 60 degrees.

14. The method according to any one of the preceding claims, wherein said individual with orthostatic intolerance or orthostatic hypotension has a sustained reduction of systolic BP of at least 20 mmHg; a sustained reduction of diastolic BP of at least 10 mmHg, and/or a sustained decrease in systolic BP to an absolute value 90 mmHg or lower after standing, or after a head-up tilt of at least 60 degrees.

15. The method according to any one of the preceding claims, wherein said individual with orthostatic intolerance has a heart rate increase of more than 30 bpm, for adults and/or a heart rate increase of more than 40 bpm for adolescents, and/or a heart rate of at least 120 bpm within 10 minutes of standing, or within a head-up tilt of at least 60 degrees.

16. The method according to any one of the preceding claims, wherein said individual with orthostatic challenges or orthostatic intolerance is a female individual.

17. The method according to any one of the preceding claims, wherein said orthostatic intolerance is postprandial orthostatic intolerance.

18. The method according to any one of the preceding claims, wherein said orthostatic intolerance is selected from the group consisting of orthostatic hypotension, such as postprandial orthostatic hypotension; reduction of blood pressure, such as postprandial reduction of blood pressure; orthostatic tachycardia, such as postprandial orthostatic tachycardia; orthostatic increase in heart rate, such as postprandial orthostatic increase in heart rate; orthostatic syncope, orthostatic presyncope, orthostatic dizziness, orthostatic light-headedness, postprandial orthostatic syncope, postprandial orthostatic presyncope, postprandial orthostatic dizziness, postprandial orthostatic light-headedness, orthostatic intolerance with hypermobility spectrum disorders/hypermobile Ehlers-Danlos syndrome (HSD/hEDS), and combinations thereof.

19. The method according to any one of the preceding claims, wherein said orthostatic intolerance is POTS.

20. The method according to any one of the preceding claims, wherein said individual is diagnosed with POTS.

21. The method according to any one of the preceding claims, wherein said orthostatic intolerance is orthostatic hypotension.

22. The method according to any one of the preceding claims, wherein said orthostatic intolerance is postprandial orthostatic hypotension.

23. The method according to any one of the preceding claims, wherein said orthostatic intolerance is orthostatic syncope.

24. The method according to any one of the preceding claims, wherein said orthostatic intolerance is postprandial orthostatic syncope.

25. method according to any one of the preceding claims, wherein said orthostatic intolerance is orthostatic intolerance with hypermobility spectrum disorders/hypermobile Ehlers-Danlos syndrome (HSD/hEDS).

26. The method according to any one of the preceding claims, wherein said GIP activity modulator is a GIP receptor (GIPR) ligand.

27. The method according to any one of the preceding claims, wherein said GIP activity modulator is a GIPR activity modulator.

28. The method according to any one of the preceding claims, wherein said GIP activity modulator or said GIPR activity modulator is a GIP and/or GIPR inhibitor.

29. The method according to any one of the preceding claims, wherein said GIP activity modulator is an agonist of a GIPR, such as hGIPR.

30. The method according to any one of the preceding claims, wherein said GIP activity modulator is an antagonist of a GIPR, such as hGIPR.

31. The method according to any one of the preceding claims, wherein said GIP activity modulator is an antibody.

32. The method according to any one of the preceding claims, wherein said GIP activity modulator is a small molecule.

33. The method according to any one of the preceding claims, wherein said GIP activity modulator is a peptide.

34. The method according to any one of the preceding claims, wherein said GIP activity modulator is a GIP antibody.

35. The method according to any one of the preceding claims, wherein said GIP activity modulator is a GIPR antibody.

36. The method according to any one of the preceding claims, wherein said GIP antibody or GIPR antibody is an antibody or an antigen-binding fragment thereof, such as an anti-GIPR antibody and/or an anti-GIPR antibody fragment or an anti-GIP antibody and/or an anti-GIP antibody fragment.

37. The method according to any one of the preceding claims, wherein said GIP antibody or GIPR antibody is a monoclonal antibody or an antigen binding fragment thereof.

38. The method according to any one of the preceding claims, wherein said GIP antibody or GIPR antibody is a polyclonal antibody or an antigen binding fragment thereof.

39. The method according to any one of the preceding claims, wherein said GIP activity modulator is an antibody selected from the group consisting of WO15095354 A2; WO15095354 A3; WO18102654 A1; WO20014329 A1; WO18124010 A1; WO21052349 A1; WO21092545 A2; WO18136440 A1; WO18169954 A1; WO17112824 A2; WO18124011 A1; WO18124009 A1; WO21202013 A1; WO16005643 A1; WO18064307 A2; WO20185533 A1; WO19179424 A1; CN114231493 A; WO21196225 A1; WO18237097 A1; WO16104439 A1; WO18237095 A1; JP2013138638 A2; WO11014680 A2; WO11014680 A3; WO06119905 A1; WO9840477 A1; WO06086823 A1; WO9824464 A1; WO03103697 A2; and WO0020592 A1.

40. The method according to any one of the preceding claims, wherein the GIP activity modulator is an agonist or an antagonist of the GIPR and an agonist or an antagonist of one or more targets such as protein targets.

41. The method according to any one of the preceding claims, wherein the GIP activity modulator is a GIP/GIPR activity modulator and one or more of a GLP-1/GLP-1R modulator, a GLP-2/GLP-2R modulator, a glucagon/glucagon receptor modulator and a Cholecystokinin (CCK) modulator.

42. The method according to any one of the preceding claims, wherein the GIP activity modulator is an agonist or an antagonist of the GIPR and an agonist or antagonist of one or more of GLP-1R, GLP-2R and glucagon receptor.

43. The method according to any one of the preceding claims, wherein the GIP activity modulator is a GIPR agonist and an agonist of GLP-1R and/or GLP-2R.

44. The method according to any one of the preceding claims, wherein the GIP activity modulator is tirzepatide.

45. The method according to any one of the preceding claims, wherein the GIP activity modulator is a GIPR agonist or GIPR antagonist selected from the group consisting of: WO17074714 A1; WO17074715 A1; WO15095354 A2; WO18102654 A1; WO20014329 A1; WO21203864 A1; WO18124010 A1; WO17116204 A1; WO17116205 A1; WO21193983 A2; WO21021877 A1; WO21260530 A1; WO21066600 A1; WO21094259 A1; WO19173787 A1; WO20096695 A1; WO20097394 A1; WO21034727 A1; WO21034728 A1; WO21068251 A1; WO21193984 A2; WO23277620 A1; WO21150673 A1; WO21239082 A1; WO07073486 A2; WO09158704 A2; WO20263063 A1; WO21145552 A1; WO22080991 A1; WO20263063 A1; WO21145552 A1; WO22080991 A1; WO21221482 A1; WO21235916 A1; WO22272018 A1; WO16108586 A1; WO21010532 A1; WO13164483 A1; WO22139538 A1; WO22257979 A1; WO15003122 A2; WO21215801 A1; WO21211976 A2; WO22066212 A1; WO22029231 A1; WO21198229 A1; WO16081884 A2; WO20023388 A1; WO21129894 A1; WO21037212 A1; WO17112824 A2; WO17116936 A1; WO11131371 A1; WO12095303 A1; WO13056852 A1; WO13104539 A1; WO13104540 A1; WO19101035 A1; WO15038938 A1; WO15095406 A1; WO18124011 A1; WO21107660 A1; WO17109087 A1; WO19211451 A1; WO18124009 A1; WO16111971 A1; WO15186988 A1; WO16066744 A2; WO20023382 A1; WO20159949 A1; WO20092191 A1; WO19245893 A2; WO22199629 A1; WO22121667 A1; WO22121666 A1; WO20130749 A1; WO22178366 A1; WO22177744 A1; WO20207477 A1; WO20207477 A9; WO21093883 A1; WO22079639 A1; WO22159395 A1; WO20244556 A1; WO18100134 A1; WO18104718 A1; WO22111370 A1; WO22035271 A1; WO22035302 A1; WO14192284 A1; WO18225041 A1; WO20185533 A1; WO13130683 A2; WO13130684 A1; CN114437181 A; WO22090447 A1; WO20067557 A2; WO15067715 A2; WO22080987 A1; WO22080986 A1; WO22080989 A1; WO19190291 A1; WO19190293 A1; WO13028989 A1; WO22065899 A1; WO22065897 A1; WO22065898 A1; CN114231493 A; WO16205488 A1; WO20023386 A1; WO20053355 A2; WO20053355 A3; CN114042149 A; WO20067575 A1; WO15067716 A1; WO21196225 A1; WO18237097 A1; WO19193204 A1; WO19229225 A1; CN113150172 A; WO21126990 A1; WO05047297 A1; WO08055940 A2; WO14096145 A1; WO14096148 A1; WO14096149 A1; WO14096150 A1; WO16049190 A1; WO13192130 A1; WO19199642 A1; WO18069442 A1; WO18237095 A1; KR20200131784 A; CN111825758 A; WO14158900 A1; WO19072963 A1; WO15184510 A1; US2017114122 AA; WO17160669 A1; WO10071807 A1; WO10016940 A2; WO11143209 A1; WO11143208 A1; WO12167744 A1; WO19140030 A1; WO10011439 A2; WO12088116 A2; WO09099763 A1; WO15086728 A1; WO13074910 A1; WO12138941 A1; WO06052608 A2; WO06105345 A2; WO06105527 A2; WO07055728 A1; WO07055743 A2; WO07114838 A1; WO13192129 A1; WO12166951 A1; WO18065634 A1; WO14081849 A1; WO15086729 A1; US2016015788 AA; WO15022420 A1; WO15086730 A1; WO10016944 A2; WO16077806 A1; WO10016938 A2; US2017112897 AA; WO10016935; WO11094337 A1; WO16198624 A1; WO16131893 A1; WO12055770 A1; WO14152460 A2; WO16077220 A1; WO15035419 A1; WO09116067 A2; WO07120689 A2; WO14049610 A2; WO13003449 A2; WO0058360 A2; WO05082928 A2; WO12168464 A1; WO14074700 A1; WO04037169 A2; CL2011003173 A1; WO06119905 A1; WO11150032 A1; WO04112701 A2; WO04064778 A2; WO05072045 A2; WO04058266 A1; WO04032836 A2; WO04110436 A1; WO06126695 A1; WO10016936 A1; WO04103276 A2; WO05094886 A1; WO0187341 A1; WO07028633 A2; WO05044195 A2; WO04069162 A2; WO9824464 A1; WO03082817 A2; WO04050022 A2; WO04050022; WO03105760 A2; WO03103697 A2; WO03082898 A2; and DOP2004000842 A.

46. The method according to any one of the preceding claims, wherein the GIP activity modulator is capable of modulating the expression of GIP or GIPR.

47. The method according to any one of the preceding claims, wherein the GIP activity modulator is an antisense oligonucleotide (ASO) or an RNAi oligonucleotide targeting GIP or a GIPR.

48. The method according to any one of the preceding claims, wherein the GIP activity modulator is a GIP or GIPR modulator selected from the group consisting of: WO18124010 A1; WO13164483 A1; WO22248506 A1; WO21198229 A1; WO18124011 A1; WO18104718 A1; CN114231493 A; CN114042149 A; WO22020388 A1; WO12161670 A2; WO21198385 A1; WO17004623 A1; WO15140212 A1; CN107385060 A; KR20190124469 A; CN103965344 A; WO08067759 A1; IN03161MU2015 A; WO08104580 A1; WO05075436 A2; WO05039548 A2; WO04098625 A2; WO04099134 A2; WO04098591 A2; GB2545395 A1; CN106676144 A; CN105219802 A; WO06086769 A2; WO08021560 A2; WO07120689 A2; WO14168153 A1; WO10122367 A2; WO10122506 A2; WO0168828 A2; CN102879767 A; WO06119905 A1; KR101091041 B1; US2009228993 AA; WO03048193 A2; WO07054577 A1; WO05094886 A1; WO05090600 A2; WO9824464 A1; AU2008200852 AA; WO05035720; WO05120474 A2; WO02096195 A1; EP1470256 A2; EP1470256 A4; WO0020592 A1; US2005159379 AA; WO03070968 A2; WO0242413 A2; JP2000342109 A2; EP0979872 A1; WO9625487 A1; EP0479210 B1; EP0269072 B1; EP0269072 A3; EP0269072 A2, WO18181864 A1; WO06025882 A2; WO10075465 A1; WO11014797 A1; WO12054500 A2; WO15095389 A1; WO19157099 A1; WO17116204 A1; WO17116205 A1; WO21193983 A2; WO21193984 A2; WO20263063 A1; WO21145552 A1; WO22080991 A1; WO21052349 A1; WO22253202 A1; WO21198229 A1; WO19101035 A1; WO19211451 A1; WO22121667 A1; WO21143810 A1; WO22159395 A1; WO20067557 A2; CN114231493 A; WO20023386 A1; WO20067575 A1; WO16049190 A1; WO18069442 A1; WO14158900 A1; WO19072963 A1; U.S. Pat. No. 10,488,423 BB; JP2019218309 A2; WO10071807 A1; WO11143209 A1; WO11143208 A1; WO12167744 A1; WO10011439 A2; WO09099763 A1; WO13074910 A1; WO12088379 A2; WO12138941 A1; WO16084826 A1; WO10148089 A1; WO11094337 A1; US2013244932 AA; WO14152460 A2; WO11104337 A1; WO06086769 A2; WO08021560 A2; WO0058360 A2; WO05082928 A2; WO08149382 A1; KR101091041 B1; WO04039392 A2; WO0020592 A1; and WO05025554 A2.

49. The method according to any one of the preceding claims, wherein the GIP activity modulator is capable of modulating GIP secretion and/or release.

50. The method according to any one of the preceding claims, wherein the GIP activity modulator is capable of inhibiting GIP secretion and/or release.

51. The method according to any one of the preceding claims, wherein the GIP activity modulator capable of inhibiting GIP secretion is selected from the group consisting of GLP-1, a GLP-1 analogue, a GLP-1R agonist and somatostatin.

52. The method according to any one of the preceding claims, wherein the GIP activity modulator capable of modulating GIP secretion and/or release is selected from the group consisting of: Semaglutide, liraglutide, dulaglutide, exenatide, WO18124010 A1; WO13164483 A1; WO18124011 A1; WO15048332 A2; WO15048333 A2; WO15048334 A2; WO15048339 A2; WO15048339 A3; WO15048340 A2; WO15048342 A2; WO15048345 A2; WO15048346 A2; WO15048348 A2; WO18124009 A1; WO16066744 A2; WO16011269 A1; WO17004432 A1; WO20205844 A1; WO21188781 A1; WO15067715 A2; JP2019043858 A2; WO12161670 A2; WO15067716 A1; WO21198385 A1; WO15141493 A1; WO10082689 A2; WO13039210 A1; WO09126719 A2; US2009143329 AA; EP2065046 A1; US2008300647 AA; WO07120689 A2; WO0058360 A2; WO05082928 A2; WO09108621 A2; CN102961388 A; JP2009126837 A2; WO07122801 A1; WO06119905 A1; JP2006342084 A2; JP2006342085 A2; WO0187341 A1; JP2009125028 A2; US2008300646 AA; US2008300645 AA; DE19921537 A1; EP0269072 A2; and EP0270321 A2.

53. The method according to any one of the preceding claims, wherein said GIP activity modulator is a GIP peptide analogue.

54. The method according to any one of the preceding claims, wherein the GIP activity modulator is a GIP peptide analogue capable of binding to and antagonising a GIPR (GIPR peptide antagonist).

55. The method according to any one of the preceding claims, wherein the GIP activity modulator is a GIP peptide analogue capable of binding to and promoting activity of a GIPR (GIPR peptide agonist).

56. The method according to any one of the preceding claims, wherein said GIP activity modulator is a GIP peptide analogue of sequence hGIP (1-42) as set forth in SEQ ID NO:1, or a functional variant thereof having at least 75% sequence identity to said hGIP (1-42).

57. The method according to any one of the preceding claims, wherein said GIP activity modulator is a GIP peptide analogue of sequence hGIP (1-42) as set forth in SEQ ID NO:1, or a functional variant thereof having 1 to 6 individual amino acid substitutions compared to said hGIP (1-42).

58. The method according to any one of the preceding claims, wherein said GIP activity modulator is a GIP peptide analogue which is a functional fragment of hGIP (1-42) (SEQ ID NO:1) wherein said functional fragment comprises 20 to 40 amino acids of said GIP (1-42); and is capable of binding a GIPR.

59. The method according to any one of the preceding claims, wherein said GIP activity modulator is a GIP peptide analogue of sequence hGIP (3-30) as set forth in SEQ ID NO: 3, or a functional variant thereof having at least 70% sequence identity to said hGIP (3-30) as set forth in SEQ ID NO: 3.

60. The method according to any one of the preceding claims, wherein said GIP activity modulator is a GIP peptide analogue of sequence hGIP (3-30) as set forth in SEQ ID NO: 3, or a functional variant thereof having 1 to 8 individual amino acid substitutions compared to said hGIP (3-30) as set forth in SEQ ID NO: 3.

61. The method according to any one of the preceding claims, wherein the GIP activity modulator is a GIP peptide analogue of sequence hGIP (3-30) as set forth in SEQ ID NO: 3, or a functional variant thereof capable of binding to a GIPR.

62. The method according to any one of the preceding claims, wherein said GIP activity modulator is a GIP peptide analogue which is a functional fragment of hGIP (3-30) as set forth in SEQ ID NO:3, wherein said functional fragment comprises 20 to 28 amino acids of said GIP (3-30); and is capable of binding a GIPR.

63. The method according to any one of the preceding claims, wherein the functional fragment comprises 22 to 28 amino acids of GIP (3-30), such as 22 to 26 amino acids, such as 22 to 24 amino acids, such as 24 to 28 amino acids, such as 24 to 26 amino acids, such as 26 to 28 amino acids of GIP (3-30).

64. The method according to any one of the preceding claims, wherein the GIP activity modulator is a GIP peptide analogue of sequence hGIP (5-30) as set forth in SEQ ID NO: 7, or a functional variant thereof capable of binding to a GIPR.

65. The method according to any one of the preceding claims, wherein said GIP activity modulator is a GIP peptide analogue which is a functional fragment of hGIP (5-30) as set forth in SEQ ID NO:7, wherein said functional fragment comprises 20 to 26 amino acids of said GIP (5-30); and is capable of binding a GIPR.

66. The method according to any one of the preceding claims, wherein the functional fragment comprises 22 to 26 amino acids of GIP (3-30), such as 22 to 26 amino acids, such as 22 to 24 amino acids, such as 20 to 24 amino acids of GIP (3-30).

67. The method according to any one of the preceding claims, wherein said GIP activity modulator is a GIP peptide analogue selected from the group consisting of hGIP (3-42) as set forth in SEQ ID NO: 2, hGIP (4-42) as set forth in SEQ ID NO: 4 and hGIP (5-42) as set forth in SEQ ID NO: 5, and a functional variant thereof having at least 75% sequence identity to any one of hGIP (3-42) as set forth in SEQ ID NO: 2, hGIP (4-42) as set forth in SEQ ID NO: 4 and hGIP (5-42) as set forth in SEQ ID NO: 5.

68. The method according to any one of the preceding claims, wherein said GIP activity modulator is a GIP peptide analogue selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant of any one of SEQ ID NOs 3 and 6-9 having at least 75% sequence identity to said peptide.

69. The method according to any one of the preceding claims, wherein said GIP activity modulator is a GIP peptide selected from the group consisting of hGIP (3-30) as set forth in SEQ ID NO: 3, hGIP (4-30) as set forth in SEQ ID NO: 6, hGIP (5-30) as set forth in SEQ ID NO: 7, hGIP (6-30) as set forth in SEQ ID NO: 8 and hGIP (7-30) as set forth in SEQ ID NO: 9, and a functional variant of any one of SEQ ID NOs 3 and 6-9 having 1 to 6 individual amino acid substitutions, such as 1, 2, 3, 4, 5 or 6 individual amino acid substitutions.

70. The method according to any one of the preceding claims, wherein said GIP activity modulator is a GIP peptide analogue which is modified by attaching at least one fatty acid molecule at one or more amino acid residues of said peptide, or a functional variant thereof.

71. The method according to any one of the preceding claims, wherein the GIP activity modulator is a GIP peptide analogue which is C-terminally amidated (—NH₂).

72. The method according to any one of the preceding claims, wherein said GIP activity modulator is a GIP peptide analogue which is C-terminally elongated.

73. The method according to claim 72, wherein said C-terminal elongation comprises one or more amino acid residues of GIP (31-42) (GKKNDWKHNITQ; SEQ ID NO: 10); or one or more amino acid residues of Exendin-4 (HGEGTFTSDLSKQMEEEAVRLFIEWLKNGGPSSGAPPPS; SEQ ID NO: 11).

74. The method according to any one of the preceding claims, wherein at least one of said at least one fatty acid molecule is a straight-chain fatty acid.

75. The method according to any one of the preceding claims, wherein at least one of said at least one fatty acid molecule is a branched fatty acid.

76. The method according to any one of the preceding claims, wherein at least one of said at least one fatty acid molecule is a monoacyl fatty acid molecule, comprising one fatty acid.

77. The method according to any one of the preceding claims, wherein at least one of said at least one fatty acid molecule is a diacyl fatty acid molecule.

78. The method according to any one of the preceding claims, wherein at least one of said at least one fatty acid molecule is attached to an amino acid residue directly.

79. The method according to any one of the preceding claims, wherein at least one of said at least one fatty acid molecule is attached to an amino acid residue via a linker.

80. The method according to any one of the preceding claims, wherein said GIP activity modulator reduces the heart rate, such as the fasting heart rate, of an individual during and/or after an orthostatic challenge.

81. The method according to any one of the preceding claims, wherein said GIP activity modulator increases the blood pressure of an individual during and/or after an orthostatic challenge.

82. The method according to any one of the preceding claims, wherein said GIP activity modulator increases the systolic blood pressure and/or the diastolic blood pressure, of an individual during and/or after an orthostatic challenge.

83. The method according to any one of the preceding claims, wherein said GIP activity modulator reduces the blood flow in superior mesenteric artery, such as reduces the fasting blood flow in superior mesenteric artery of an individual during and/or after an orthostatic challenge.

84. The method according to any one of the preceding claims, wherein said GIP activity modulator inhibits GIP activity at least 80%, such as at least 85%, such as at least 90%, such as at least 95%, such as about 100%.

85. The method according to any one of the preceding claims, wherein said GIP activity modulator inhibits GIPR activity at least 80%, such as at least 85%, such as at least 90%, such as at least 95%, such as about 100%; such as wherein said inhibition of GIPR activity is determined as a decrease in intracellular cAMP.

86. The method according to any one of the preceding claims, wherein said GIP activity modulator is capable of one or more of the following:
a. binding to a GIPR,
b. activating a GIPR,
c. antagonizing a GIPR,
d. inhibiting or reducing GIPR downstream signalling, such as cAMP generation,
e. decreasing intracellular cAMP,
f. disrupting the interaction between GIP and GIPR,
g. increasing or decreasing GIP secretion,
h. inhibiting or reducing agonist-mediated responses of GIPR, and/or
i. inhibiting GIPR activity at least 80%, such as at least 85%, such as at least 90%, such as at least 95%, such as about 100%.

87. A method of treating orthostatic intolerance, the method comprising one or more steps of administering a pharmaceutical composition, such as a pharmaceutically acceptable composition, comprising a GIP activity modulator according to any of the preceding claims and a pharmaceutically acceptable carrier, and further comprising one or more steps of administering one or more additional therapeutic agents for use in the treatment of orthostatic intolerance.
